(19) United States
(12) Patent Application Publication
(10) Pub. No.: US 2004/0106868 A1
(43) Pub. Date: Jun. 3, 2004

(54) Novel imaging markers in musculoskeletal disease

(76) Inventors: Siau-Way Liew, Pinole, CA (US); Konstantinos Tsougarakis, Mountain View, CA (US); Claude D. Arnaud, Mill Valley, CA (US); Philipp Lang, Lexington, MA (US); Daniel Steines, Palo Alto, CA (US); Barry J. Linder, Danville, CA (US)

Correspondence Address: IMAGING THERAPEUTICS, INC., C/O ROBBINS & PASTERNAK, 1731 EMBARCADERO ROAD, SUITE 230, PALO ALTO, CA, 94304-3303, US

(21) Appl. No.: 10/665,725

(22) Filed: Sep. 16, 2003

Related U.S. Application Data
(60) Provisional application No. 60/411,413, filed on Sep. 16, 2002.

(51) Int. Cl. A61B 8/00

(52) U.S. Cl. 600/442

(57) ABSTRACT

This invention is directed to methods for using imaging methods to aid in drug discovery, and drug development. This invention also relates to methods of using imaging methods for diagnosis, prognostication, monitoring and patient management of musculoskeletal disease.

### CROSS REFERENCE TO RELATED APPLICATIONS

This application is related to U.S. Provisional Patent Application Serial No. 60/411,413 filed on Sep. 16, 2002, from which priority is claimed under 35 USC '119(e)(1), and is incorporated herein by reference in its entirety.

### TECHNICAL FIELD

This invention relates to using imaging methods to aid in drug discovery and drug development. This invention also relates to using imaging methods for diagnosis, prognostication, monitoring and management of disease, particularly where that disease affects the musculoskeletal system. This invention identifies novel imaging markers for use in diagnosis, prognostication, monitoring and management of disease, including musculoskeletal disease.

### BACKGROUND

Osteoporosis and osteoarthritis are among the most common conditions to affect the musculoskeletal system, as well as frequent causes of locomotor pain and disability. Osteoporosis can occur in both human and animal subjects (e.g. horses). Osteoporosis (OP) and osteoarthritis (OA) occur in a substantial portion of the human population over the age of fifty. The National Osteoporosis Foundation (www.nof.org) estimates that as many as 44 million Americans are affected by osteoporosis and low bone mass. In 1997 the estimated cost for osteoporosis related fractures was $13 billion. That figure increased to $17 billion in 2002 and is projected to increase to $210-240 billion by 2040. Currently it is expected that one in two women over the age of 50 will suffer an osteoporosis-related fracture.

In spite of its societal impact and prevalence, there is a paucity of information on the factors that cause osteoporosis and osteoarthritis to progress more rapidly in some individuals than others. Previously considered diseases with little opportunity for therapeutic intervention, osteoporosis and osteoarthritis are now increasingly viewed as a dynamic process with potential for new pharmacologic and surgical treatment modalities.

However, the appropriate deployment and selection of treatment interventions for OP and OA is dependent on the development of better methods for the assessment of the condition of a patient. Moreover, better methods for the assessment of the condition of a patient can also help with drug discovery, drug development, diagnosis, prognostication, disease monitoring, therapeutic monitoring and patient management.

The present invention discloses novel methods and techniques for assessing the condition of a bone(s) and/or a joint(s).

### SUMMARY OF THE INVENTION

The invention discloses a method for analyzing at least one of bone mineral density, bone structure and surrounding tissue. The method typically comprises: (a) obtaining an image of a subject; (b) locating a region of interest on the image; (c) obtaining data from the region of interest; and (d) deriving data selected from the group of qualitative and quantitative from the image data obtained at step c.

Further, a kit is provided for aiding in the assessment of the condition of at least one of a bone and joint. The kit typically comprises a software program which reads at least one of a degeneration pattern, a pattern of normal tissue, a pattern of abnormal tissue, and a pattern of diseased tissue. The kit can also include a database of measurements for comparison to the at least one of degeneration pattern, pattern of normal tissue, pattern of abnormal tissue, and pattern of diseased tissue. Additionally, the kit can include a subset of a database of measurements for comparison to the at least one of degeneration pattern, pattern of normal tissue, pattern of abnormal tissue, and pattern of diseased tissue.

The invention also includes at least one of an automated and semi-automated method of using an imaging marker. This automatic or semi-automatic method comprises: obtaining image data from a subject; obtaining data from the image data wherein the data obtained is at least one of quantitative and qualitative data; and administering an agent. Either the automated or semi-automated method can be used for: drug discovery, diagnosis, disease staging, disease monitoring, disease management, prognostication, therapy monitoring, drug efficacy monitoring, and disease prediction.

In another embodiment, a system for monitoring the efficacy of an agent and/or a system for drug discovery is provided. The system includes: administering an agent to a subject; obtaining image data; and obtaining data from the image data wherein the data obtained is at least one of quantitative and qualitative data.

A system is also provided for diagnosing a disease, determining disease staging, monitoring disease progression, managing a disease, disease prognostication, predicting a disease, monitoring therapy and/or randomizing a subject within a group of patients comprising. Any of these systems can include the steps of: (a) obtaining image data of a subject; (b) obtaining data from the image data wherein the data obtained is at least one of quantitative and qualitative data; and (c) comparing the at least one of quantitative and qualitative data in step b to at least one of: a database of at least one of quantitative and qualitative data obtained from a group of subjects; at least one of quantitative and qualitative data obtained from the subject; and at least one of a quantitative and qualitative data obtained from the subject at time Tn.

In any of these described invention, additional steps can be provided. Such additional steps include, for example, enhancing image data.

Suitable subjects for these steps include for example mammals, humans and horses. Suitable anatomical regions of subjects include, for example, dental, spine, hip, knee and bone core x-rays.

A variety of systems can be employed to practice the inventions. Typically at least one of the steps of any of the methods is performed on a first computer. Although, it is possible to have an arrangement where at least one of the steps of the method is performed on a first computer and at least one of the steps of the method is performed on a second computer. In this scenario the first computer and the second computer are typically connected. Suitable connections include, for example, a peer to peer network, direct link, intranet, and internet.

It is important to note that any or all of the steps of the inventions disclosed can be repeated one or more times in series or in parallel with or without the repetition of other steps in the various methods. This includes, for example repeating the step of locating a region of interest, or obtaining image data.

Data can also be converted from 2D to 3D to 4D and back; or from 2D to 4D. Data conversion can occur at multiple points of processing the information. For example, data conversion can occur before or after pattern evaluation and/or analysis.

Any of the processes described herein are suitable for including the step of administering a candidate agent, where that step has not already been performed. Suitable agents include, for example, substances administered to a subject, substances ingested by a subject, molecules, pharmaceuticals, biopharmaceuticals, agropharmaceuticals, genetically manufactured and altered substances, etc.

Any data obtained, extracted or generated under any of the methods can be compared to a database, a subset of a database, or data previously obtained, extracted or generated from the subject.

The present invention provides methods that allow for the analysis of bone mineral density, bone and/or cartilage structure and morphology and/or surrounding tissue from images including electronic images and, accordingly, allows for the evaluation of the effect(s) of an agent (or agents) on bone and/or cartilage. It is important to note that an effect on bone and/or cartilage can occur in agents intended to have an effect, such as a therapeutic effect, on bone and/or cartilage as well as agents intended to primarily effect other tissues in the body but which have a secondary, or tangential, effect on bone and/or cartilage. The images (e.g., x-ray images) can be, for example, dental, hip, spine or other radiographs and can be taken from any mammal. The images can be in electronic format.

The invention includes a method to derive quantitative information on bone structure and/or bone mineral density from an image comprising (a) obtaining an image, wherein the image optionally includes an external standard for determining bone density and/or structure; and (b) analyzing the image obtained in step (a) to derive quantitative information on bone structure. The image is taken of a region of interest (ROI). Suitable ROI include, for example, a hip radiograph or a dental x-ray obtained on dental x-ray film, including the mandible, maxilla or one or more teeth. In certain embodiments, the image is obtained digitally, for example using a selenium detector system, a silicon detector system or a computed radiography system. In other embodiments, the image can be digitized from film, or another suitable source, for analysis.

A method is included where one or more candidate agents can be tested for its effects on bone. Again, the effect can be a primary effect or a secondary effect. For example, the candidate agent can be administered to a subject; thereafter an electronic image of a portion of a bone of the subject can be obtained; and finally the image obtained can be analyzed for information on bone structure. Information on bone structure can relate to a variety of parameters, including the parameters shown in Table 1, Table 2 and Table 3, infra. The images or data can then be compared to a database of images or data (e.g., “normal” images or data) and/or compared to one or more images or data taken from the same subject prior to administration of the candidate agent or to one or more images or data obtained in a reference population. The candidate agent can, for example, be molecules, proteins, peptides, naturally occurring substances, chemically synthesized substances, or combinations and cocktails thereof. Typically, an agent is one or more drugs. Further, the agent can be evaluated for the ability to effect bone diseases such as the risk of bone fracture (e.g., osteoporotic fracture).

In any of the methods described herein, the analysis can comprise using one or more computer programs (or units). Additionally, the analysis can comprise identifying one or more regions of interest (ROI) in the image, either prior to, concurrently or after analyzing the image, e.g. for information on bone mineral density and/or bone structure. The bone density information can be, for example, areas of highest, lowest or median density. Bone structural information can be, for example, one or more of the parameters shown in Table 1, Table 2 and Table 3. The various analyses can be performed concurrently or in series. Further, when using two or more indices each of the indices can be weighted equally or differently, or combinations thereof where more than two indices are employed. Additionally, any of these methods can also include analyzing the image for bone mineral density information using any of the methods described herein.

Any of the methods described herein can further comprise applying one or more correction factors to the data obtained from the image. For example, correction factors can be programmed into a computer unit. The computer unit can be the same one that performs the analysis of the image or can be a different unit. In certain embodiments, the correction factors account for the variation in soft-tissue thickness in individual subjects.

These and other embodiments of the subject invention will readily occur to those of skill in the art in light of the disclosure herein.

### BRIEF DESCRIPTION OF THE DRAWINGS

FIGS. 1A AND B are block diagrams showing the steps for extracting data from an image and then deriving quantitative and/or qualitative data from the image.

FIGS. 2A-C are diagrams showing an image taken of a region of anatomical interest further illustrating possible locations of regions of interest for analysis.

FIGS. 3A-J illustrate various abnormalities that might occur including, for example, cartilage defects, bone marrow edema, subchondral sclerosis, osteophytes and cysts.

FIGS. 4A AND B are block diagrams of the method of FIG. 1A showing that the steps can be repeated.

FIGS. 5A-E are block diagrams illustrating steps involved in evaluating patterns in an image of a region of interest.

FIGS. 6A-E are block diagrams illustrating steps involved in deriving quantitative and qualitative data from an image in conjunction with administering candidate molecules or drugs for evaluation.

FIGS. 7A-D are block diagrams illustrating steps involved in comparing derived quantitative and qualitative information to a database or to information obtained at a previous time.

FIGS. 8A-D are block diagrams illustrating steps involved in comparing converting an image to a pattern of normal and diseased tissue

FIG. 9 is a diagram showing the use one or more devices in the process of developing a degeneration pattern and using a database for degeneration patterns.

### DETAILED DESCRIPTION

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all issued patents, patent publications, and patent applications cited in this application are incorporated herein by reference.

The practice of the present invention employs, unless otherwise indicated, currently conventional methods of imaging and image processing within the skill of the art. Such techniques are explained fully in the literature. See, e.g., WO 02/22014, X-Ray Structure Determination: A Practical Guide, 2^{nd} Edition, editors Stout and Jensen, 1989, John Wiley & Sons, publisher; Body CT: A Practical Approach, editor Slone, 1999, McGraw-Hill publisher; The Essential Physics of Medical Imaging, editors Bushberg, Seibert, Leidholdt Jr & Boone, 2002, Lippincott, Williams & Wilkins; X-ray Diagnosis: A Physician's Approach, editor Lam, 1998 Springer-Verlag, publisher; Dental Radiology: Understanding the X-Ray Image, editor Laetitia Brocklebank 1997, Oxford University Press publisher; and Digital Image Processing, editor Kenneth R. Castleman, 1996 Prentice Hall, publisher; The Image Processing Handbook, editor John C. Russ, 3^{rd} Edition, 1998, CRC Press; Active Contours: The Application of Techniques from Graphics, Vision, Control Theory and Statistics to Visual Tracking of Shapes in Motion, Editors Andrew Blake, Michael Isard, 1999 Springer Verlag. As will be appreciated by those of skill in the art, as the field of imaging continues to advance methods of imaging currently employed can evolve over time. Thus, any imaging method or technique that is currently employed is appropriate for application of the teachings of this invention as well as techniques that can be developed in the future. A further detailed description of imaging methods is not provided in order to avoid obscuring the invention.

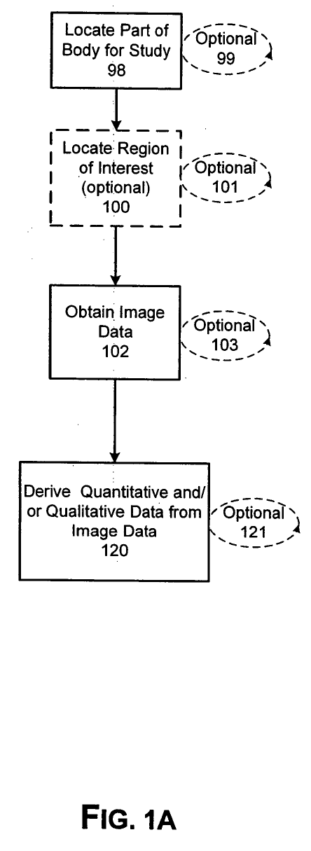

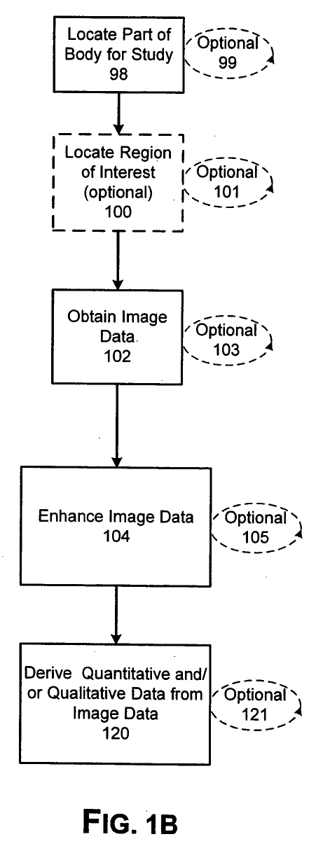

As shown in FIG. 1A, the first step is to locate a part of the body of a subject, for example in a human body, for study 98. The part of the body located for study is the region of anatomical interest (RAI). In locating a part of the body for study, a determination is made to, for example, take an image or a series of images of the body at a particular location, e.g. hip, dental, spine, etc. Images include, for example, conventional x-ray images, x-ray tomosynthesis, ultrasound (including A-scan, B-scan and C-scan) computed tomography (CT scan), magnetic resonance imaging (MRI), optical coherence tomography, single photon emission tomography (SPECT), and positron emission tomography, or such other imaging tools that a person of skill in the art would find useful in practicing the invention. Once the image is taken, a region of interest (ROI) can be located within the image 100. Image data is extracted from the image 102. Finally, quantitative and/or qualitative data is extracted from the image data 120. The quantitative and/or qualitative data extracted from the image includes, for example, the parameters and measurements shown in Table 1, Table 2 or Table 3.

Each step of locating a part of the body for study 98, optionally locating a region of interest 100, obtaining image data 102, and deriving data 120, can be repeated one or more times 99,101, 103, 121, respectively, as desired.

As shown in FIG. 1B image data can be optionally enhanced 104 by applying image processing techniques, such as noise filtering or diffusion filtering, to facilitate further analysis. Similar to the process shown in FIG. 1A, locating a part of the body for study 98, optionally locating a region of interest 100, obtaining image data 102, enhancing image data 104, and deriving data 120, can be repeated one or more times 99,101, 103, 105, 121, respectively, as desired.

TABLE 1
Representative Parameters Measured with
Quantitative and Qualitative Image Analysis Methods

| PARAMETER | MEASUREMENTS |
|---|---|
| Bone | Stainless steel equivalent thickness |
| parameters | (Average gray value of the region of interest expressed |
| | as thickness of stainless steel with equivalent intensity) |
| | Trabecular contrast |
| | (Trabecular equivalent thickness/Marrow equivalent |
| | thickness) |
| | Fractal dimension |
| | Fourier spectral analysis |
| | (Mean transform coefficient absolute value and mean |
| | spatial first moment) |
| | Predominant orientation of spatial energy spectrum |
| | Trabecular area |
| | (Pixel count of extracted trabeculae) |
| | Trabecular area/Total area |
| | Trabecular perimeter |
| | (Count of trabecular pixels with marrow pixels in their |
| | neighborhood, proximity or vicinity) |
| | Trabecular distance transform |
| | (For each trabecular pixel, calculation of distance to |
| | closest marrow pixel) |
| | Marrow distance transform |
| | (For each marrow pixel, calculation of distance to closest |
| | trabecular pixel) |
| | Trabecular distance transform regional maximal values |
| | (mean, min., max, std. Dev). |
| | (Describes thickness and thickness variation of |
| | trabeculae) |
| | Marrow distance transform regional maximal values |
| | (mean, min., max, std. Dev) |
| | Star volume |
| | (Mean volume of all the parts of an object which can |
| | be seen unobscured from a random point inside the object |
| | in all possible directions) |
| | Trabecular Bone Pattern Factor |
| | (TBPf = (P1 − P2)/(A1 − A2) where P1 and A1 are the |
| | perimeter length and trabecular bone area before dilation |
| | and P2 and A2 corresponding values after a single pixel |
| | dilation, measure of connectivity) |
| | Connected skeleton count or Trees (T) |
| | Node count (N) |
| | Segment count (S) |
| | Node-to-node segment count (NN) |
| | Node-to-free-end segment count (NF) |
| | Node-to-node segment length (NNL) |
| | Node-to-free-end segment length (NFL) |
| | Free-end-to-free-end segment length (FFL) |
| | Node-to-node total struts length (NN.TSL) |
| | Free-end-to-free-ends total struts length (FF.TSL) |
| | Total struts length (TSL) |
| | FF.TSL/TSL |
| | NN.TSL/TSL |
| | Loop count (Lo) |
| | Loop area |
| | Mean distance transform values for each connected |
| | skeleton |
| | Mean distance transform values for each segment |
| | (Tb.Th) |
| | Mean distance transform values for each node-to-node |
| | segment (Tb.Th.NN) |
| | Mean distance transform values for each node-to-free-end |
| | segment (Tb.Th.NF) |
| | Orientation (angle) of each segment |
| | Angle between segments |
| | Length-thickness ratios (NNL/Tb.Th.NN) and (NFL/ |
| | Tb.Th.NF) |
| | Interconnectivity index (ICI) ICI = (N * NN)/ |
| | (T * (NF + 1)) |
| Cartilage and | Total cartilage volume |
| cartilage | Partial/Focal cartilage volume |
| defect/diseased | Cartilage thickness distribution (thickness map) |
| cartilage | Mean cartilage thickness for total region or focal region |
| parameters | Median cartilage thickness for total region or focal region |
| | Maximum cartilage thickness for total region or focal |
| | region |
| | Minimum cartilage thickness for total region or focal |
| | region |
| | 3D cartilage surface information for total region or focal |
| | region |
| | Cartilage curvature analysis for total region or focal |
| | region |
| | Volume of cartilage defect/diseased cartilage |
| | Depth of cartilage defect/diseased cartilage |
| | Area of cartilage defect/diseased cartilage |
| | 2D or 3D location of cartilage defect/diseased cartilage |
| | in articular surface |
| | 2D or 3D location of cartilage defect/diseased cartilage in |
| | relationship to weight-bearing area |
| | Ratio: diameter of cartilage defect or diseased cartilage/ |
| | thickness of surrounding normal cartilage |
| | Ratio: depth of cartilage defect or diseased cartilage/ |
| | thickness of surrounding normal cartilage |
| | Ratio: volume of cartilage defect or diseased cartilage/ |
| | thickness of surrounding normal cartilage |
| | Ratio: surface area of cartilage defect or diseased |
| | cartilage/total joint or articular surface area |
| | Ratio: volume of cartilage defect or diseased cartilage/ |
| | total cartilage volume |
| Other articular | Presence or absence of bone marrow edema |
| parameters | Volume of bone marrow edema |
| | Volume of bone marrow edema normalized by width, |
| | area, size, volume of femoral condyle(s)/tibial plateau/ |
| | patella - other bones in other joints |
| | Presence or absence of osteophytes |
| | Presence or absence of subchondral cysts |
| | Presence or absence of subchondral sclerosis |
| | Volume of osteophytes |
| | Volume of subchondral cysts |
| | Volume of subchondral sclerosis |
| | Area of bone marrow edema |
| | Area of osteophytes |
| | Area of subchondral cysts |
| | Area of subchondral sclerosis |
| | Depth of bone marrow edema |
| | Depth of osteophytes |
| | Depth of subchondral cysts |
| | Depth of subchondral sclerosis |
| | Volume, area, depth of osteophytes, subchondral cysts, |
| | subchondral sclerosis normalized by width, area, size, |
| | volume of femoral condyle(s)/tibial plateau/patella - other |
| | bones in other joints |
| | Presence or absence of meniscal tear |
| | Presence or absence of cruciate ligament tear |
| | Presence or absence of collateral ligament tear |
| | Volume of menisci |
| | Ratio of volume of normal to torn/damaged or |
| | degenerated meniscal tissue |
| | Ratio of surface area of normal to torn/damaged or |
| | degenerated meniscal tissue |
| | Ratio of surface area of normal to torn/damaged or |
| | degenerated meniscal tissue to total joint or cartilage |
| | surface area |
| | Ratio of surface area of torn/damaged or degenerated |
| | meniscal tissue to total joint or cartilage surface area |
| | Size ratio of opposing articular surfaces |
| | Meniscal subluxation/dislocation in mm |
| | Index combining different articular parameters which can |
| | also include |
| | Presence or absence of cruciate or collateral |
| | ligament tear |
| | Body mass index, weight, height |
| | 3D surface contour information of subchondral bone |
| | Actual or predicted knee flexion angle during gait cycle |
| | (latter based on gait patterns from subjects with matching |
| | demographic data retrieved from motion profile database) |
| | Predicted knee rotation during gait cycle |
| | Predicted knee displacement during gait cycle |
| | Predicted load bearing line on cartilage surface during |
| | gait cycle and measurement of distance between load |
| | bearing line and cartilage defect/diseased cartilage |
| | Predicted load bearing area on cartilage surface during |
| | gait cycle and measurement of distance between load |
| | bearing area and cartilage defect/diseased cartilage |
| | Predicted load bearing line on cartilage surface during |
| | standing or different degrees of knee flexion and |
| | extension and measurement of distance between load |
| | bearing line and cartilage defect/diseased cartilage |
| | Predicted load bearing area on cartilage surface during |
| | standing or different degrees of knee flexion and |
| | extension and measurement of distance between load |
| | bearing area and cartilage defect/diseased cartilage |
| | Ratio of load bearing area to area of cartilage defect/ |
| | diseased cartilage |
| | Percentage of load bearing area affected by cartilage |
| | disease |
| | Location of cartilage defect within load bearing area |
| | Load applied to cartilage defect, area of diseased |
| | cartilage |
| | Load applied to cartilage adjacent to cartilage defect, area |
| | of diseased cartilage |

As will be appreciated by those of skill in the art, the parameters and measurements shown in Table 1 are provided for illustration purposes. Other parameters and measurements, ratios, derived values or indices can be used to extract quantitative and/or qualitative information about the ROI without departing from the scope of the invention. Additionally, where multiple ROI or multiple derivatives of data are used, the parameter measured can be the same parameter or a different parameter without departing from the scope of the invention. Additionally, data from different ROI's can be combined or compared as desired.

Additional measurements can be performed that are selected based on the anatomical structure to be studied as described below.

Once the data is extracted from the image it can be manipulated to assess the severity of the disease and to determine disease staging (e.g., mild, moderate, severe or a numerical value or index). The information can also be used to monitor progression of the disease and/or the efficacy of any interventional steps that have been taken. Finally, the information can be used to predict the progression of the disease or to randomize patient groups in clinical trials.

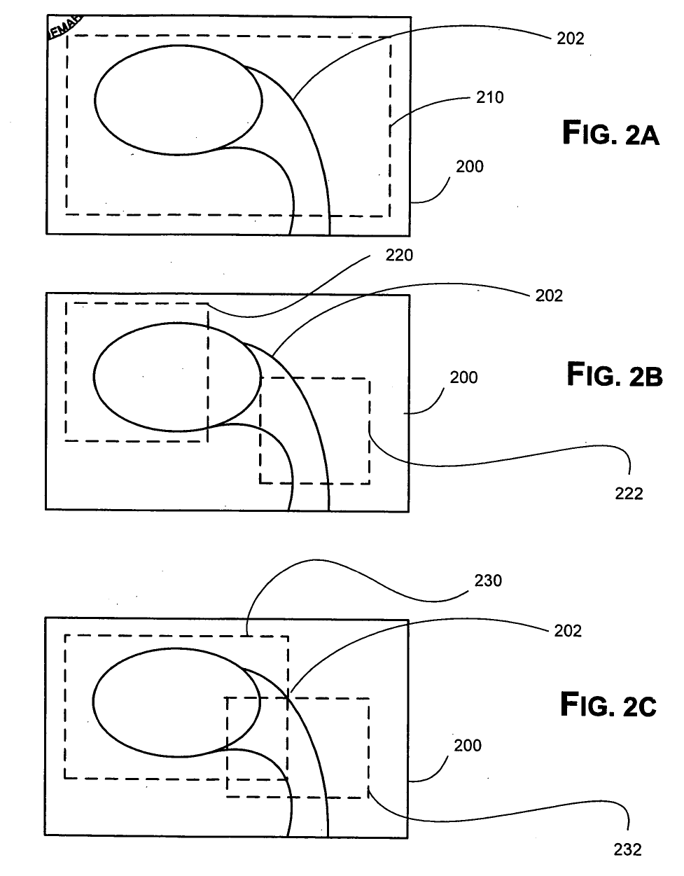

FIG. 2A illustrates an image 200 taken of an RAI, shown as 202. As shown in FIG. 2A, a single region of interest (ROI) 210 has been identified within the image. The ROI 210 can take up the entire image 200, or nearly the entire image. As shown in FIG. 2B more than one ROI can be identified in an image. In this example, a first ROI 220 is depicted in one region of the image 200, and a second ROI 222 is depicted within the image. In this instance, neither of these ROI overlap or abut. As will be appreciated by a person of skill in the art, the number of ROI identified in an image 200 is not limited to the two depicted. Turning now to FIG. 2c another embodiment showing two ROI for illustration purposes is shown. In this instance, the first ROI 230 and the second ROI 232, are partially overlapping. As will be appreciated by those of skill in the art, where multiple ROI are used any or all of the ROI can be organized such that it does not overlap, it abuts without overlapping, it overlaps partially, it overlaps completely (for example where a first ROI is located completely within a second identified ROI), and combinations thereof. Further the number of ROI per image 200 can range from one (ROI₁) to n (ROIₙ) where n is the number of ROI to be analyzed.

Bone density, microarchitecture, macro-anatomic and/or biomechanical (e.g. derived using finite element modeling) analyses can be applied within a region of predefined size and shape and position. This region of interest can also be referred to as a “window.” Processing can be applied repeatedly within the window at different positions of the image. For example, a field of sampling points can be generated and the analysis performed at these points. The results of the analyses for each parameter can be stored in a matrix space, e.g., where its position corresponds to the position of the sampling point where the analysis occurred, thereby forming a map of the spatial distribution of the parameter (a parameter map). The sampling field can have regular intervals or irregular intervals with varying density across the image. The window can have variable size and shape, for example to account for different patient size or anatomy.

The amount of overlap between the windows can be determined, for example, using the interval or density of the sampling points (and resolution of the parameter maps). Thus, the density of sampling points is set higher in regions where higher resolution is desired and set lower where moderate resolution is sufficient, in order to improve processing efficiency. The size and shape of the window would determine the local specificity of the parameter. Window size is preferably set such that it encloses most of the structure being measured. Oversized windows are generally avoided to help ensure that local specificity is not lost.

The shape of the window can be varied to have the same orientation and/or geometry of the local structure being measured to minimize the amount of structure clipping and to maximize local specificity. Thus, both 2D and/or 3D windows can be used, as well as combinations thereof, depending on the nature of the image and data to be acquired.

In another embodiment, bone density, microarchitecture, macro-anatomic and/or biomechanical (e.g. derived using finite element modeling) analyses can be applied within a region of predefined size and shape and position. The region is generally selected to include most, or all, of the anatomic region under investigation and, preferably, the parameters can be assessed on a pixel-by-pixel basis (e.g., in the case of 2D or 3D images) or a voxel-by-voxel basis in the case of cross-sectional or volumetric images (e.g., 3D images obtained using MR and/or CT). Alternatively, the analysis can be applied to clusters of pixels or voxels wherein the size of the clusters is typically selected to represent a compromise between spatial resolution and processing speed. Each type of analysis can yield a parameter map.

Parameter maps can be based on measurement of one or more parameters in the image or window; however, parameter maps can also be derived using statistical methods. In one embodiment, such statistical comparisons can include comparison of data to a reference population, e.g. using a z-score or a T-score. Thus, parameter maps can include a display of z-scores or T-scores.

Additional measurements relating to the site to be measured can also be taken. For example, measurements can be directed to dental, spine, hip, knee or bone cores. Examples of suitable site specific measurements are shown in Table 2.

TABLE 2
Site specific measurement of bone parameters

| | |
|---|---|
| Parameters | All microarchitecture parameters on structures |
| specific to | parallel to stress lines |
| hip x-rays | All microarchitecture parameters on structures |
| | perpendicular to stress lines |

Geometry

Shaft angle
Neck angle
Diameter of femur neck
Hip axis length
Largest cross-section of femur head
Average thickness of cortical within ROI
Standard deviation of cortical thickness within ROI
Maximum thickness of cortical within ROI
Minimum thickness of cortical within ROI

Hip joint space width

| | |
|---|---|
| Parameters | All microarchitecture parameters on vertical structures |
| specific to | All microarchitecture parameters on horizontal |
| spine x-rays | structures |

Geometry

| | |
|---|---|
| | Superior endplate cortical thickness (anterior, center, |
| | posterior) |
| | Inferior endplate cortical thickness (anterior, center, |
| | posterior) |
| | Anterior vertebral wall cortical thickness (superior, |
| | center, inferior) |
| | Posterior vertebral wall cortical thickness (superior, |
| | center, inferior) |
| | Superior aspect of pedicle cortical thickness |
| | inferior aspect of pedicle cortical thickness |
| | Vertebral height (anterior, center, posterior) |
| | Vertebral diameter (superior, center, inferior), |
| | Pedicle thickness (supero-inferior direction). |
| | Maximum vertebral height |
| | Minimum vertebral height |
| | Average vertebral height |
| | Anterior vertebral height |
| | Medial vertebral height |
| | Posterior vertebral height |
| | Maximum inter-vertebral height |
| | Minimum inter-vertebral height |
| | Average inter-vertebral height |
| Parameters | Average medial joint space width |
| specific to | Minimum medial joint space width |
| knee x-rays | Maximum medial joint space width |
| | Average lateral joint space width |
| | Minimum lateral joint space width |
| | Maximum lateral joint space width |

As will be appreciated by those of skill in the art, measurement and image processing techniques are adaptable to be applicable to both microarchitecture and macro-anatomical structures. Examples of these measurements are shown in Table 3.

TABLE 3
Measurements applicable on Microarchitecture and Macro-anatomical
Structures

| | |
|---|---|
| Average density | Calibrated density of ROI |
| measurement | |

| | |
|---|---|
| Measurements on | The following parameters are derived from the |
| micro-anatomical | extracted structures: |

| | |
|---|---|
| structures of | Calibrated density of extracted structures |
| dental, spine, hip, | Calibrated density of background |
| knee or bone | Average intensity of extracted structures |
| cores x-rays | Average intensity of background (area other than |
| | extracted structures) |
| | Structural contrast (average intensity of extracted |
| | structures/average intensity of background) |
| | Calibrated structural contrast (calibrated density |
| | extracted structures/calibrated density of |
| | background) |
| | Total area of extracted structures |
| | Total area of ROI |
| | Area of extracted structures normalized by total |
| | area of ROI |
| | Boundary lengths (perimeter) of extracted |
| | normalized by total area of ROI |
| | Number of structures normalized by area of ROI |
| | Trabecular bone pattern factor; measures |
| | concavity and convexity of structures |
| | Star volume of extracted structures |
| | Star volume of background |
| | Number of loops normalized by area of ROI |

| | |
|---|---|
| Measurements on | The following statistics are measured from the |
| Distance | distance transform regional maximum values: |

| | |
|---|---|
| transform of | Average regional maximum thickness |
| extracted | Standard deviation of regional maximum |
| structures | thickness |
| | Largest value of regional maximum thickness |
| | Median of regional maximum thickness |
| Measurements on | Average length of networks (units of connected |
| skeleton of | segments) |
| extracted | Maximum length of networks |
| structures | Average thickness of structure units (average |
| | distance transform values along skeleton) |
| | Maximum thickness of structure units (maximum |
| | distance transform values along skeleton) |
| | Number of nodes normalized by ROI area |
| | Number of segments normalized by ROI area |
| | Number of free-end segments normalized by ROI |
| | area |
| | Number of inner (node-to-node) segments |
| | normalized ROI area |
| | Average segment lengths |
| | Average free-end segment lengths |
| | Average inner segment lengths |
| | Average orientation angle of segments |
| | Average orientation angle of inner segments |
| | Segment tortuosity; a measure of straightness |
| | Segment solidity; another measure of straightness |
| | Average thickness of segments (average distance |
| | transform values along skeleton segments) |
| | Average thickness of free-end segments |
| | Average thickness of inner segments |
| | Ratio of inner segment lengths to inner segment |
| | thickness |
| | Ratio of free-end segment lengths to free-end |
| | segment thickness |
| | Interconnectivity index; a function of number of |
| | inner segments, free-end segments and number of |
| | networks. |

| | |
|---|---|
| Directional | All measurement of skeleton segments can be |
| skeleton | constrained by one or more desired orientation by |
| segment | measuring only skeleton segments within ranges |
| measurements | of angle. |
| Watershed | Watershed segmentation is applied to gray level |
| segmentation | images. |
| | Statistics of watershed segments are: |

Total area of segments
Number of segments normalized by total area of
segments
Average area of segments
Standard deviation of segment area
Smallest segment area
Largest segment area

Calibrated density typically refers to the measurement of intensity values of features in images converted to its actual material density or expressed as the density of a reference material whose density is known. The reference material can be metal, polymer, plastics, bone, cartilage, etc., and can be part of the object being imaged or a calibration phantom placed in the imaging field of view during image acquisition.

Extracted structures typically refer to simplified or amplified representations of features derived from images. An example would be binary images of trabecular patterns generated by background subtraction and thresholding. Another example would be binary images of cortical bone generated by applying an edge filter and thresholding. The binary images can be superimposed on gray level images to generate gray level patterns of structure of interest.

Distance transform typically refers to an operation applied on binary images where maps representing distances of each 0 pixel to the nearest 1 pixel are generated. Distances can be calculated by the Euclidian magnitude, city-block distance, La Place distance or chessboard distance.

Distance transform of extracted structures typically refer to distance transform operation applied to the binary images of extracted structures, such as those discussed above with respect to calibrated density.

Skeleton of extracted structures typically refer to a binary image of 1 pixel wide patterns, representing the centerline of extracted structures. It is generated by applying a skeletonization or medial transform operation, by mathematical morphology or other methods, on an image of extracted structures.

Skeleton segments typically are derived from skeleton of extracted structures by performing pixel neighborhood analysis on each skeleton pixel. This analysis classifies each skeleton pixel as a node pixel or a skeleton segment pixel. A node pixel has more than 2 pixels in its 8-neighborhood. A skeleton segment is a chain of skeleton segment pixels continuously 8-connected. Two skeleton segments are separated by at least one node pixel.

Watershed segmentation as it is commonly known to a person of skill in the art, typically is applied to gray level images to characterize gray level continuity of a structure of interest. The statistics of dimensions of segments generated by the process are, for example, those listed in Table 3 above. As will be appreciated by those of skill in the art, however, other processes can be used without departing from the scope of the invention.

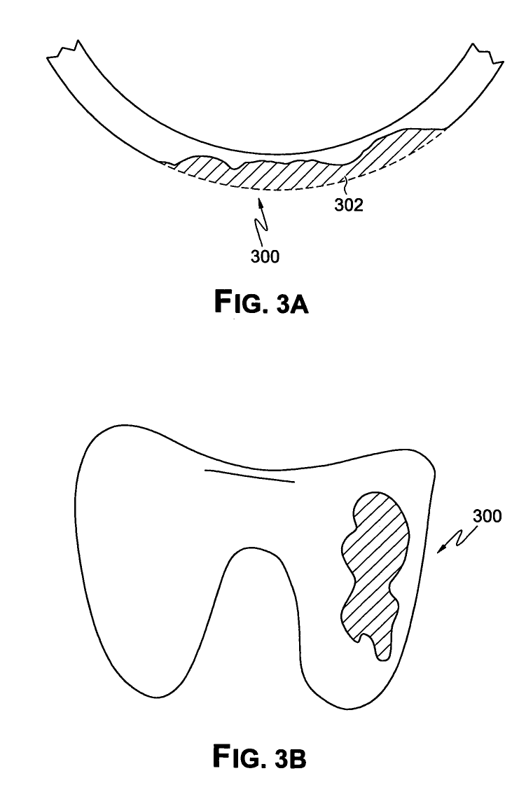

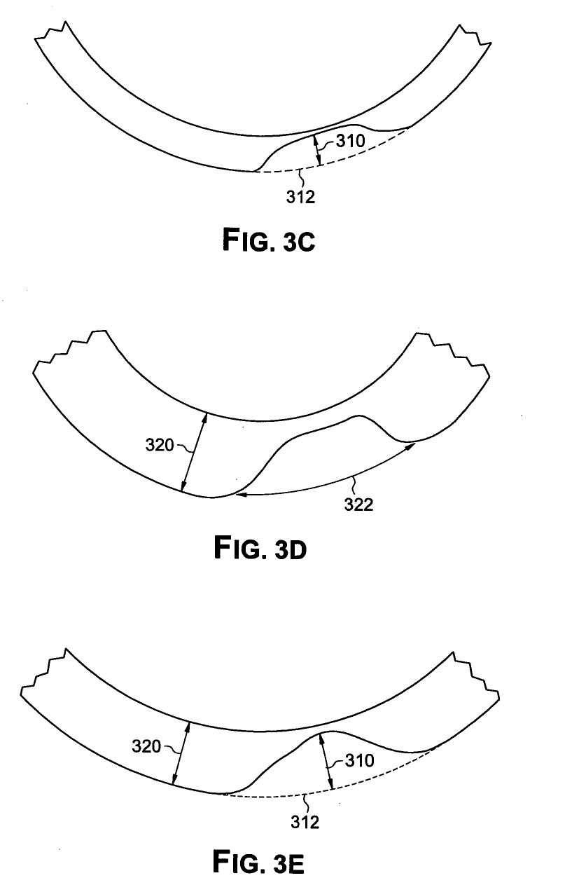

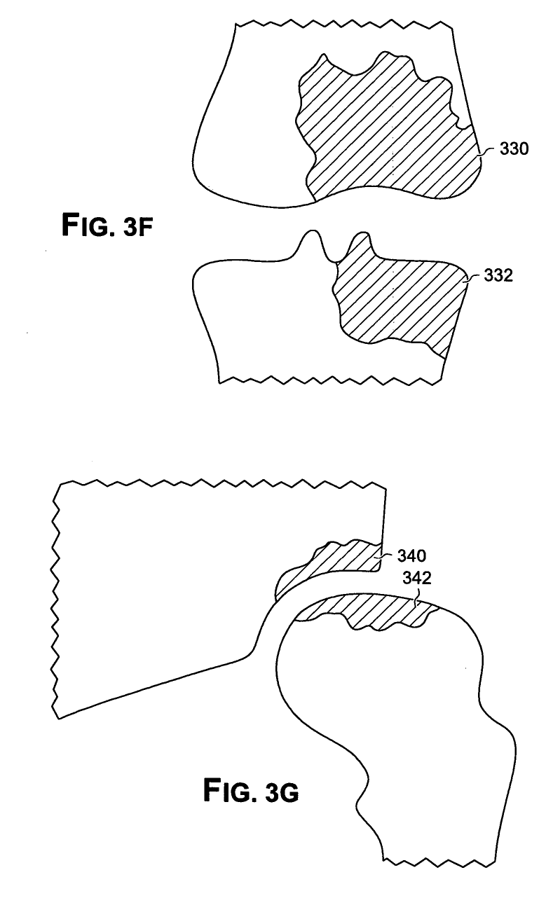

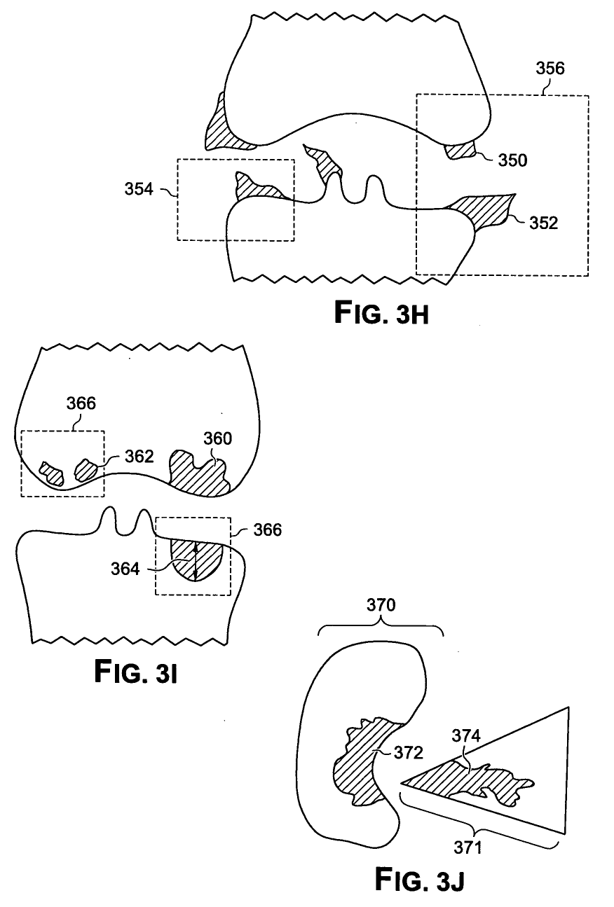

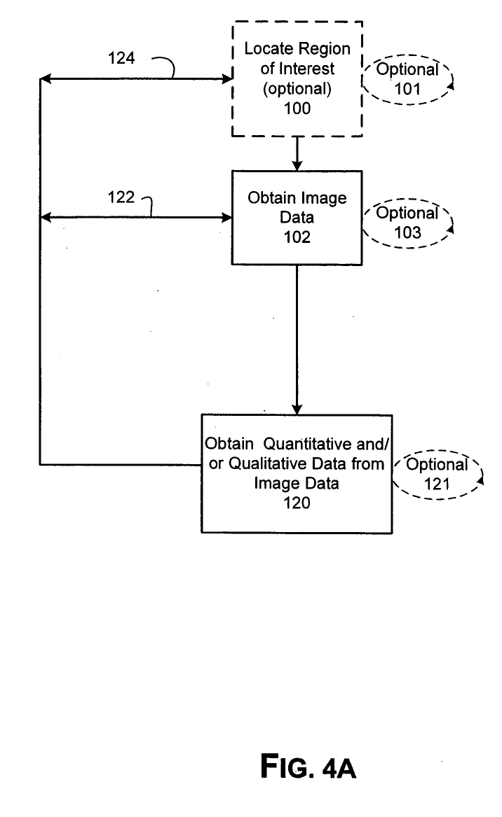

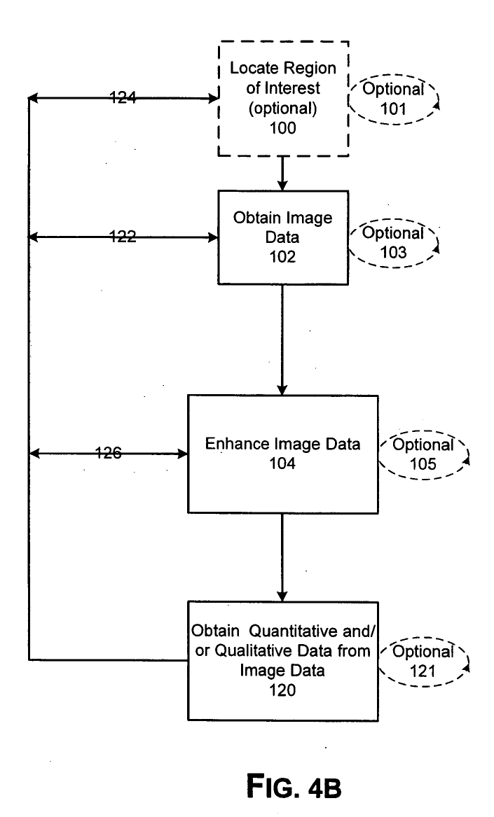

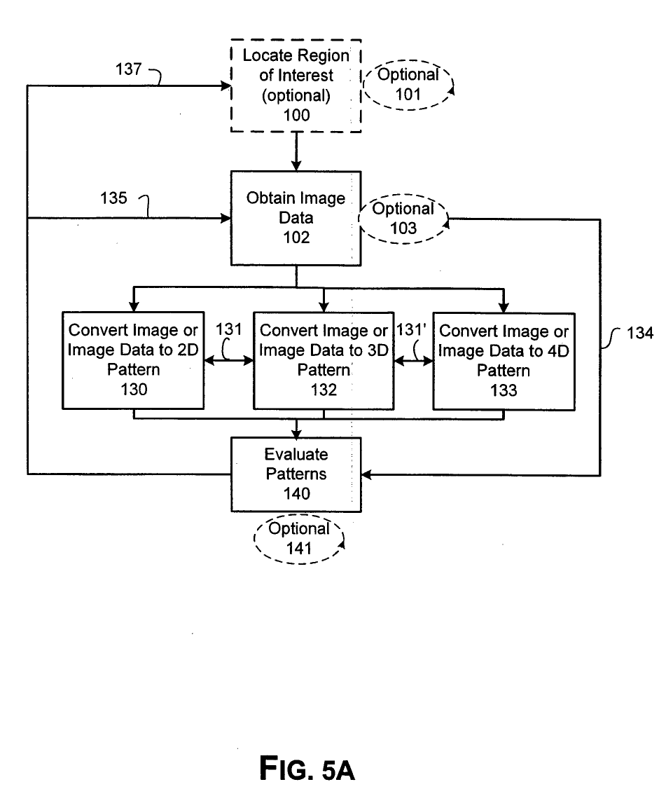

Turning now to FIG. 3A, a cross-section of a cartilage defect is shown 300. The cross-hatched zone 302 corresponds to an area where there is cartilage loss. FIG. 3B is a top view of the cartilage defect shown in FIG. 3A.

FIG. 3c illustrates the depth of a cartilage defect 310 in a first cross-section dimension with a dashed line illustrating a projected location of the original cartilage surface 312. By comparing these two values a ratio of cartilage defect depth to cartilage defect width can be calculated.

FIG. 3D illustrated the depth of the cartilage 320 along with the width of the cartilage defect 322. These two values can be compared to determine a ratio of cartilage depth to cartilage defect width.

FIG. 3E shows the depth of the cartilage defect 310 along with the depth of the cartilage 320. A dashed line is provided illustrating a projected location for the original cartilage surface 312. Similar to the measurements made above, ratios between the various measurements can be calculated.

Turning now to FIG. 3F, an area of bone marrow edema is shown on the femur 330 and the tibia 332. The shaded area of edema can be measured on a T2-weighted MRI scan. Alternatively, the area can be measured on one or more slices. These measurements can then be extended along the entire joint using multiple slices or a 3D acquisition. From these measurements volume can be determined or derived.

FIG. 3G shows an area of subchondral sclerosis in the acetabulum 340 and the femur 342. The sclerosis can be measured on, for example, a T1 or T2-weighted MRI scan or on a CT scan. The area can be measured on one or more slices. Thereafter the measurement can be extended along the entire joint using multiple slices or a 3D acquisition. From these values a volume can be derived of the subchondral sclerosis. For purposes of illustration, a single sclerosis has been shown on each surface. However, a person of skill in the art will appreciate that more than one sclerosis can occur on a single joint surface.

FIG. 3H shows osteophytes on the femur 350 and the tibia 352. The osteophytes are shown as cross-hatched areas. Similar to the sclerosis shown in FIG. 3G, the osteophytes can be measured on, for example, a T1 or T2-weighted MRI scan or on a CT scan. The area can be measured on one or more slices. Thereafter the measurement can be extended along the entire joint using multiple slices or a 3D acquisition. From these values a volume can be derived of the osteophytes. Additionally, a single osteophyte 354 or osteophyte groups 356 can be included in any measurement. Persons of skill in the art will appreciate that groups can be taken from a single joint surface or from opposing joint surfaces, as shown, without departing from the scope of the invention.

Turning now to FIG. 31 an area of subchondral cysts 360, 362, 364 is shown. Similar to the sclerosis shown in FIG. 3G, the cysts can be measured on, for example, a T1 or T2-weighted MRI scan or on a CT scan. The area can be measured on one or more slices. Thereafter the measurement can be extended along the entire joint using multiple slices or a 3D acquisition. From these values a volume can be derived of the cysts. Additionally, single cysts 366 or groups of cysts 366′ can be included in any measurement. Persons of skill in the art will appreciate that groups can be taken from a single joint surface, as shown, or from opposing joint surfaces without departing from the scope of the invention.

FIG. 3J illustrates an area of torn meniscal tissue (cross-hatched) 372, 374 as seen from the top 370 and in cross-section 371. Again, similar to the sclerosis shown in FIG. 3G, the torn meniscal tissue can be measured on, for example, a T1 or T2-weighted MRI scan or on a CT scan. The area can be measured on one or more slices. Thereafter the measurement can be extended along the entire joint using multiple slices or a 3D acquisition. From these values a volume can be derived of the tear. Ratios such as surface or volume of torn to normal meniscal tissue can be derived as well as ratios of surface of torn meniscus to surface of opposing articulating surface.

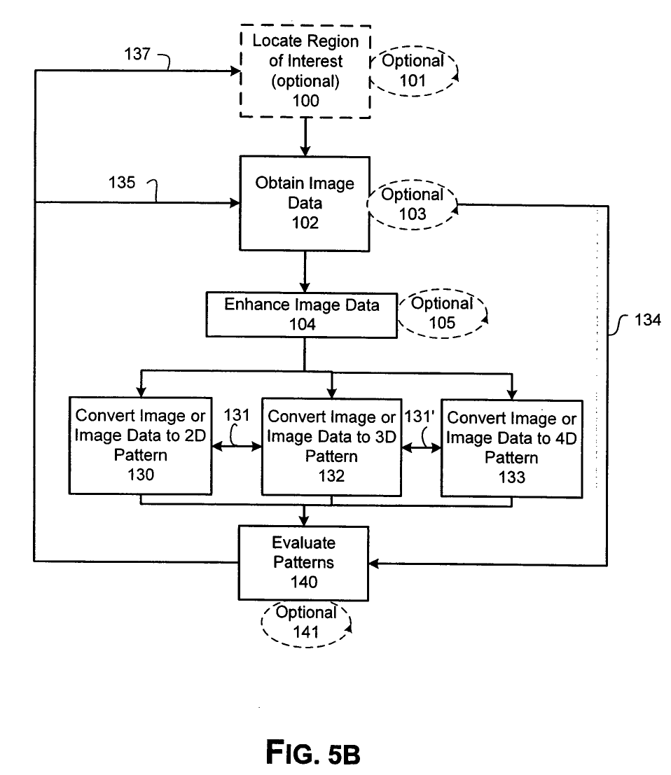

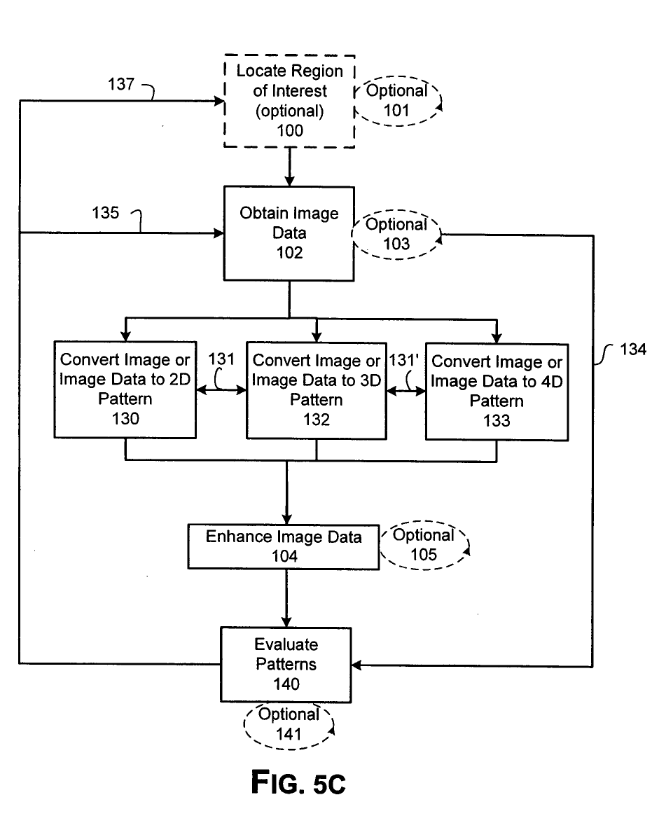

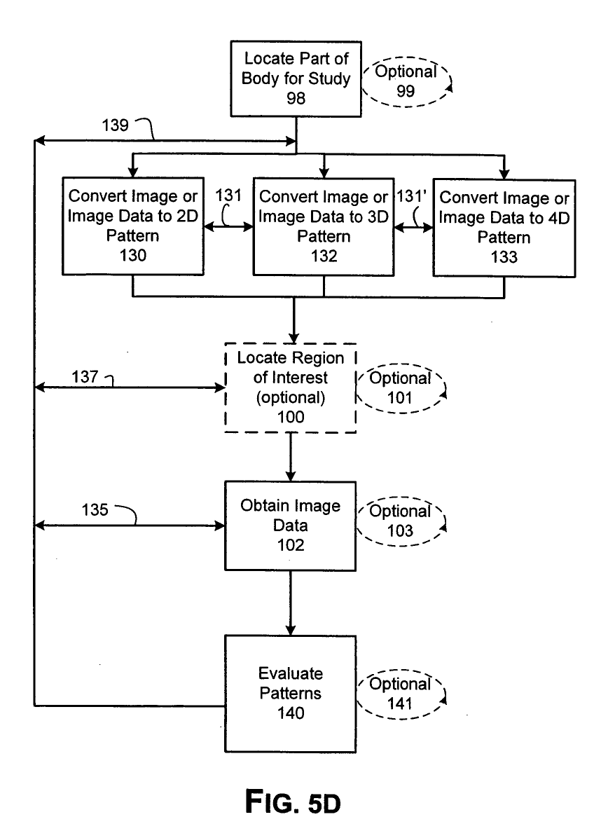

As shown in FIG. 4A, the process of optionally locating a ROI 100, extracting image data from the ROI 102, and deriving quantitative and/or qualitative image data from the extracted image data 120, can be repeated 122. Alternatively, or in addition, the process of locating a ROI 100, can be repeated 124. A person of skill in the art will appreciate that these steps can be repeated one or more times in any appropriate sequence, as desired, to obtain a sufficient amount of quantitative and/or qualitative data on the ROI or to separately extract or evaluate parameters. Further, the ROI used can be the same ROI as used in the first process or a newly identified ROI in the image. Additionally, as with FIG. 1A the steps of locating a region of interest 100, obtaining image data 102, and deriving quantitative and/or qualitative image data can be repeated one or more times, as desired, 101, 103, 121, respectively. Although not depicted here, as discussed above with respect to FIG. 1A, the additional step of locating a part of the body for study 98 can be performed prior to locating a region of interest 100 without departing from the invention. Additionally that step can be repeated 99.

FIG. 4B illustrates the process shown in FIG. 4A with the additional step enhancing image data 104. Additionally, the step of enhancing image data 104 can be repeated one or more times 105, as desired. The process of enhancing image data 104 can be repeated 126 one or more times as desired.

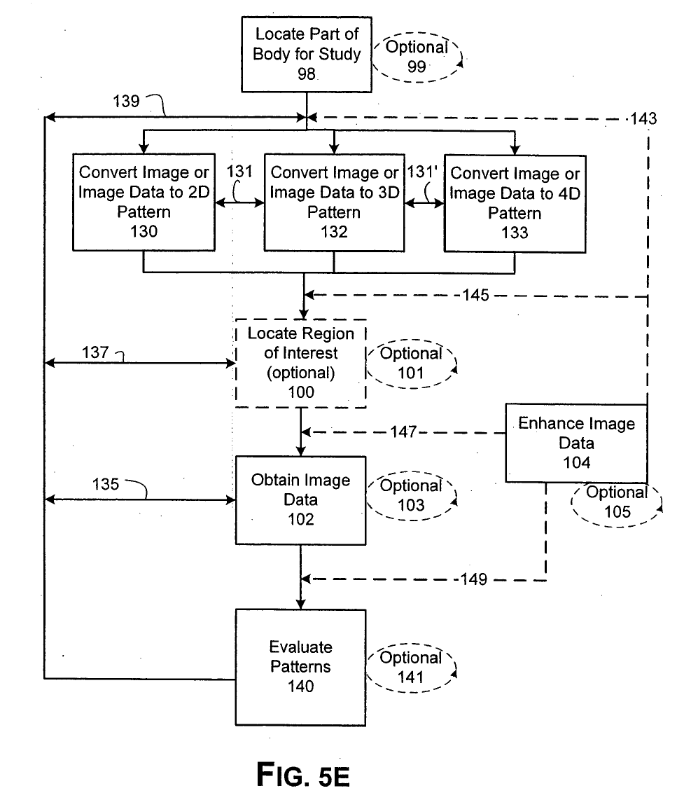

Turning now to FIG. 5A, a process is shown whereby a region of interest is optionally located 100. Although not depicted here, as discussed above with respect to FIG. 1A, the step of locating a part of the body for study 98 can be performed prior to locating a region of interest 100 without departing from the invention. Additionally that step can be repeated 99. Once the region of interest is located 100, and image data is extracted from the ROI 102, the extracted image data can then be converted to a 2D pattern 130, a 3D pattern 132 or a 4D pattern 133, for example including velocity or time, to facilitate data analyses. Following conversion to 2D 130, 3D 132 or 4D pattern 133 the images are evaluated for patterns 140. Additionally images can be converted from 2D to 3D 131, or from 3D to 4D 131′, if desired. Although not illustrated to avoid obscuring the figure, persons of skill in the art will appreciate that similar conversions can occur between 2D and 4D in this process or any process illustrated in this invention.

As will be appreciated by those of skill in the art, the conversion step is optional and the process can proceed directly from extracting image data from the ROI 102 to evaluating the data pattern 140 directly 134. Evaluating the data for patterns, includes, for example, performing the measurements described in Table 1, Table 2 or Table 3, above.

Additionally, the steps of locating the region of interest 100, obtaining image data 102, and evaluating patterns 141 can be performed once or a plurality of times, 101, 103, 141, respectively at any stage of the process. As will be appreciated by those of skill in the art, the steps can be repeated. For example, following an evaluation of patterns 140, additional image data can be obtained 135, or another region of interest can be located 137. These steps can be repeated as often as desired, in any combination desirable to achieve the data analysis desired.

FIG. 5B illustrates an alternative process to that shown in FIG. 5A which includes the step of enhancing image data 104 prior to converting an image or image data to a 2D 130, 3D 132, or 4D 133 pattern. The process of enhancing image data 104, can be repeated 105 if desired. FIG. 5c illustrates an alternative embodiment to the process shown in FIG. 5B. In this process, the step of enhancing image data 104 occurs after converting an image or image data to a 2D 130, 3D 132, or 4D 133 pattern. Again, the process of enhancing image data 104, can be repeated 105 if desired.

FIG. 5D illustrates an alternative process to that shown in FIG. 5A. After locating a part of the body for study 98 and imaging, the image is then converted to a 2D pattern 130, 3D pattern 132 or 4D pattern 133. The region of interest 100 is optionally located within the image after conversion to a 2D, 3D or 4D image and data is then extracted 102. Patterns are then evaluated in the extracted image data 140. As with the process of FIG. 5A, the conversion step is optional. Further, if desired, images can be converted between 2D, 3D 131 and 4D 131′ if desired.

Similar to FIG. 5A, some or all the processes can be repeated one or more times as desired. For example, locating a part of the body for study 98, locating a region of interest 100, obtaining image data 102, and evaluating patterns 140, can be repeated one or more times if desired, 99, 101, 103, 141, respectively. Again steps can be repeated. For example, following an evaluation of patterns 140, additional image data can be obtained 135, or another region of interest can be located 137 and/or another portion of the body can be located for study 139. These steps can be repeated as often as desired, in any combination desirable to achieve the data analysis desired.

FIG. 5E illustrates an alternative process to that shown in FIG. 5D. In this process image data can be enhanced 104. The step of enhancing image data can occur prior to conversion 143, prior to locating a region of interest 145, prior to obtaining image data 102, or prior to evaluating patterns 149.

Similar to FIG. 5A, some or all the processes can be repeated one or more times as desired, including the process of enhancing image data 104, which is shown as 105.

The method also comprises obtaining an image of a bone or a joint, optionally converting the image to a two-dimensional or three-dimensional or four-dimensional pattern, and evaluating the amount or the degree of normal, diseased or abnormal tissue or the degree of degeneration in a region or a volume of interest using one or more of the parameters specified in Table 1, Table 2 and/or Table 3. By performing this method at an initial time T₁, information can be derived that is useful for diagnosing one or more conditions or for staging, or determining, the severity of a condition. This information can also be useful for determining the prognosis of a patient, for example with osteoporosis or arthritis. By performing this method at an initial time T₁, and a later time T₂, the change, for example in a region or volume of interest, can be determined which then facilitates the evaluation of appropriate steps to take for treatment. Moreover, if the subject is already receiving therapy or if therapy is initiated after time T₁, it is possible to monitor the efficacy of treatment. By performing the method at subsequent times, T₂-Tₙ, additional data ca be acquired that facilitate predicting the progression of the disease as well as the efficacy of any interventional steps that have been taken. As will be appreciated by those of skill in the art, subsequent measurements can be taken at regular time intervals or irregular time intervals, or combinations thereof. For example, it can be desirable to perform the analysis at T₁ with an initial follow-up, T₂, measurement taken one month later. The pattern of one month follow-up measurements could be performed for a year (12 one-month intervals) with subsequent follow-ups performed at 6 month intervals and then 12 month intervals. Alternatively, as an example, three initial measurements could be at one month, followed by a single six month follow up which is then followed again by one or more one month follow-ups prior to commencing 12 month follow ups. The combinations of regular and irregular intervals are endless, and are not discussed further to avoid obscuring the invention.

Moreover, one or more of the parameters listed in Tables 1, 2 and 3 can be measured. The measurements can be analyzed separately or the data can be combined, for example using statistical methods such as linear regression modeling or correlation. Actual and predicted measurements can be compared and correlated.

The method for assessing the condition of a bone or joint in a subject can be fully automated such that the measurements of one or more of the parameters specified in Table 1, Table 2 or Table 3 are done automatically without intervention. The automatic assessment then can include the steps of diagnosis, staging, prognostication or monitoring the disease or diseases, or to monitor therapy. As will be appreciated by those of skill in the art, the fully automated measurement is, for example, possible with image processing techniques such as segmentation and registration. This process can include, for example, seed growing, thresholding, atlas and model based segmentation methods, live wire approaches, active and/or deformable contour approaches, contour tracking, texture based segmentation methods, rigid and non-rigid surface or volume registration, for example based on mutual information or other similarity measures. One skilled in the art will readily recognize other techniques and methods for fully automated assessment of the parameters and measurements specified in Table 1, Table 2 and Table 3.

Alternatively, the method of assessing the condition of a bone or joint in a subject can be semi-automated such that the measurements of one or more of the parameters, such as those specified in Table 1, are performed semi-automatically, i.e., with intervention. The semi-automatic assessment then allows for human interaction and, for example, quality control, and utilizing the measurement of said parameter(s) to diagnose, stage, prognosticate or monitor a disease or to monitor a therapy. The semi-automated measurement is, for example, possible with image processing techniques such as segmentation and registration. This can include seed growing, thresholding, atlas and model based segmentation methods, live wire approaches, active and/or deformable contour approaches, contour tracking, texture based segmentation methods, rigid and non-rigid surface or volume registration, for example base on mutual information or other similarity measures. One skilled in the art will readily recognize other techniques and methods for semi-automated assessment of the parameters specified in Table 1, Table 2 or Table 3.

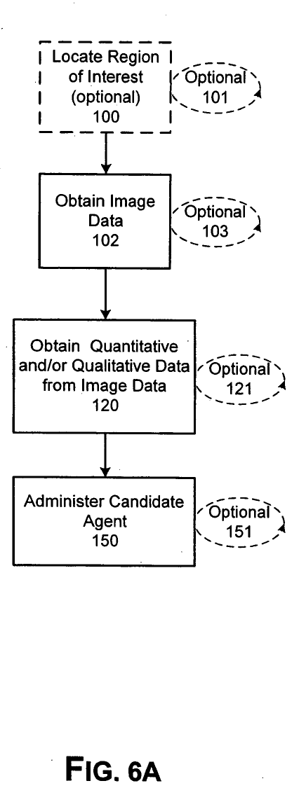

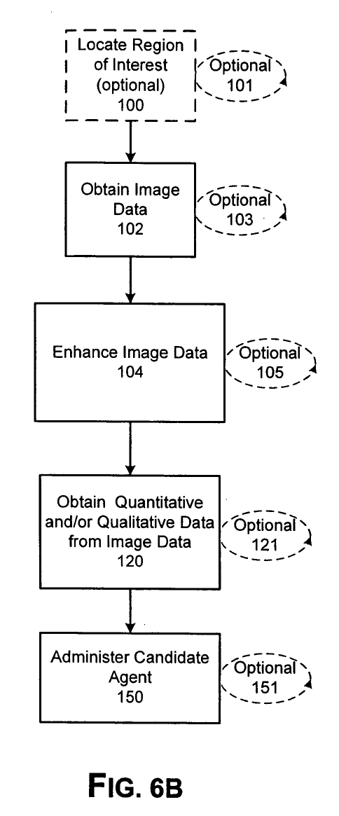

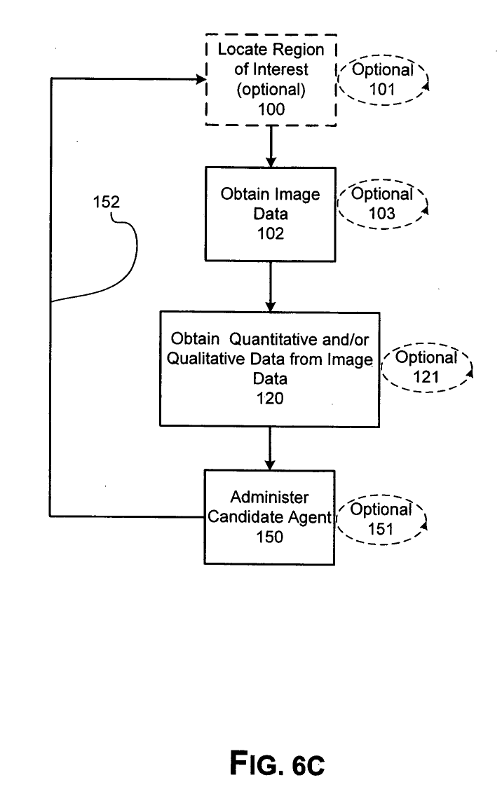

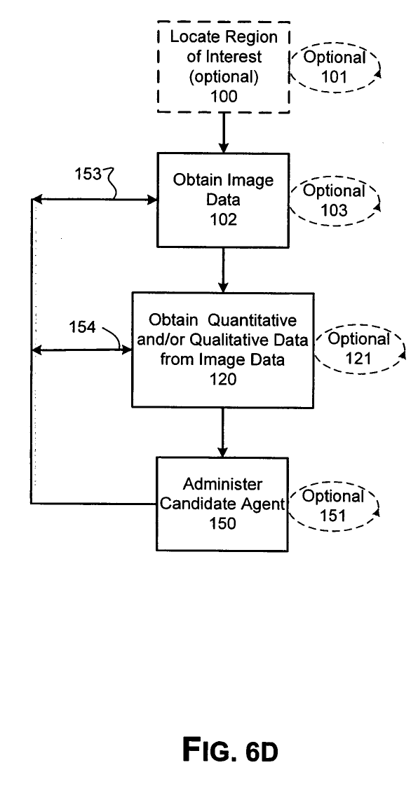

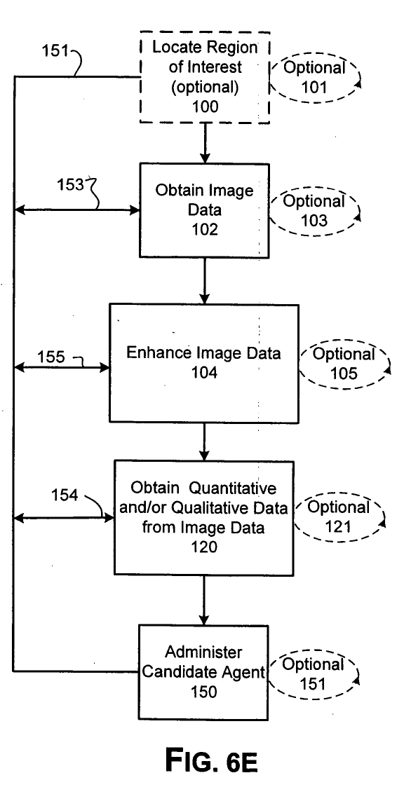

Turning now to FIG. 6A, a process is shown whereby the user locates a ROI 100, extracts image data from the ROI 102, and then derives quantitative and/or qualitative image data from the extracted image data 120, as shown above with respect to FIG. 1. Following the step of deriving quantitative and/or qualitative image data, a candidate agent is administered to the patient 150. The candidate agent can be any agent the effects of which are to be studied. Agents can include any substance administered or ingested by a subject, for example, molecules, pharmaceuticals, biopharmaceuticals, agropharmaceuticals, or combinations thereof, including cocktails, that are thought to affect the quantitative and/or qualitative parameters that can be measured in a region of interest. These agents are not limited to those intended to treat disease that affects the musculoskeletal system but this invention is intended to embrace any and all agents regardless of the intended treatment site. Thus, appropriate agents are any agents whereby an effect can be detected via imaging. The steps of locating a region of interest 100, obtaining image data 102, obtaining quantitative and/or qualitative data from image data 120, and administering a candidate agent 150, can be repeated one or more times as desired, 101, 103, 121, 151, respectively.

FIG. 6B shows the additional step of enhancing image data 104, which can also be optionally repeated 105 as often as desired.

As shown in FIG. 6c these steps can be repeated one or more times 152 to determine the effect of the candidate agent. As will be appreciated by those of skill in the art, the step of repeating can occur at the stage of locating a region of interest 152 as shown in FIG. 6B or it can occur at the stage obtaining image data 153 or obtaining quantitative and/or qualitative data from image data 154 as shown in FIG. 6D.

FIG. 6E shows the additional step of enhancing image data 104, which can optionally be repeated 105, as desired.

As previously described, some or all the processes shown in FIGS. 6A-E can be repeated one or more times as desired. For example, locating a region of interest 100, obtaining image data 102, enhancing image data 104, obtaining quantitative and/or qualitative data 120, evaluating patterns 140, and administering candidate agent 150 can be repeated one or more times if desired, 101, 103, 105, 121, 141, 151 respectively.

In the scenario described in relation to FIG. 6, an image is taken prior to administering the candidate agent. However, as will be appreciated by those of skill in the art, it is not always possible to have an image prior to administering the candidate agent. In those situations, progress is determined over time by evaluating the change in parameters from extracted image to extracted image.

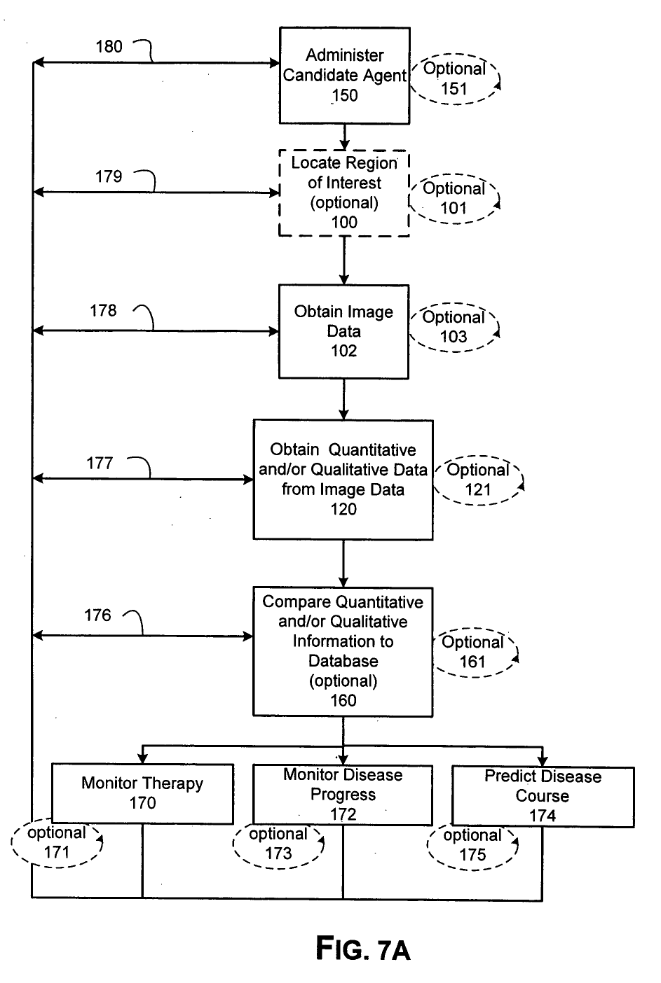

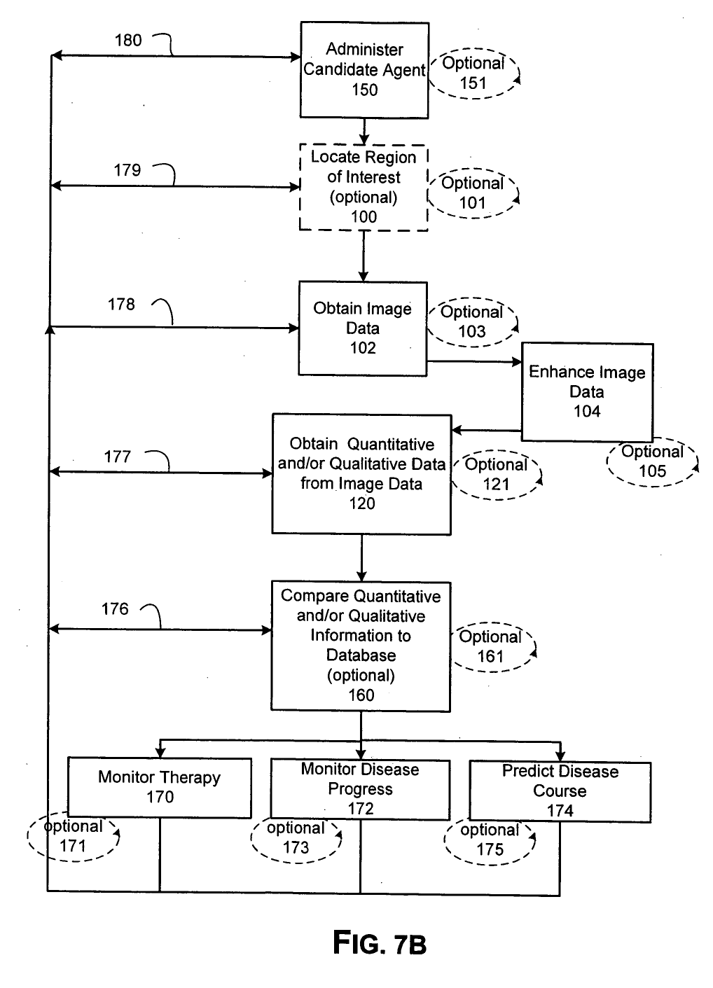

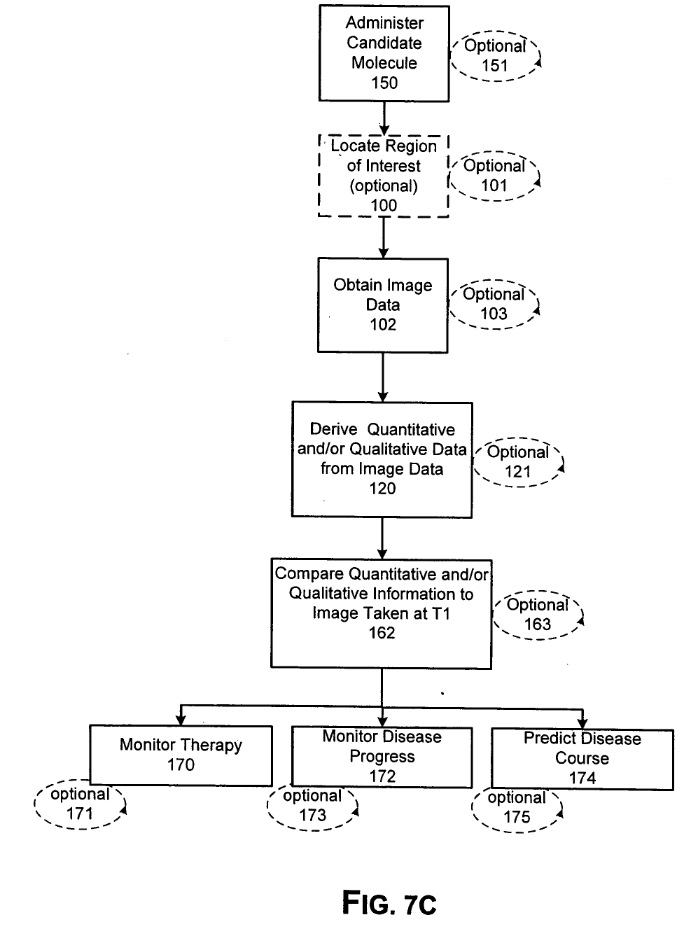

Turning now to FIG. 7A, the process is shown whereby the candidate agent is administered first 150. Thereafter a region of interest is located in an image taken 100 and image data is extracted 102. Once the image data is extracted, quantitative and/or qualitative data is extracted from the image data 120. In this scenario, because the candidate agent is administered first, the derived quantitative and/or qualitative data derived is compared to a database 160 or a subset of the database which includes data for subjects having similar tracked parameters. As shown in FIG. 7B following the step of obtaining image data, the image data can be enhanced 104. This process can optionally be repeated 105, as desired.

Alternatively, as shown in FIG. 7c the derived quantitative and/or qualitative information can be compared to an image taken at T1 162, or any other time, if such image is available. As shown in FIG. 7D the step of enhancing image data 104 can follow the step of obtaining image data 102. Again, the process can be repeated 105, as desired.

As previously described, some or all the processes illustrated in FIGS. 7A-D can be repeated one or more times as desired. For example, locating a region of interest 100, obtaining image data 102, enhancing image data 104, obtaining quantitative and/or qualitative data 120, administering candidate agent 150, comparing quantitative and/or qualitative information to a database 160, comparing quantitative and/or qualitative information to an image taken at a prior time, such as T₁, 162, monitoring therapy 170, monitoring disease progress 172, predicting disease course 174 can be repeated one or more times if desired, 101, 103, 105, 121, 151, 161, 163, 171, 173, 175 respectively. Each of these steps can be repeated in one or more loops as shown in FIG. 7B, 176, 177, 178, 179, 180, as desired or appropriate to enhance data collection.

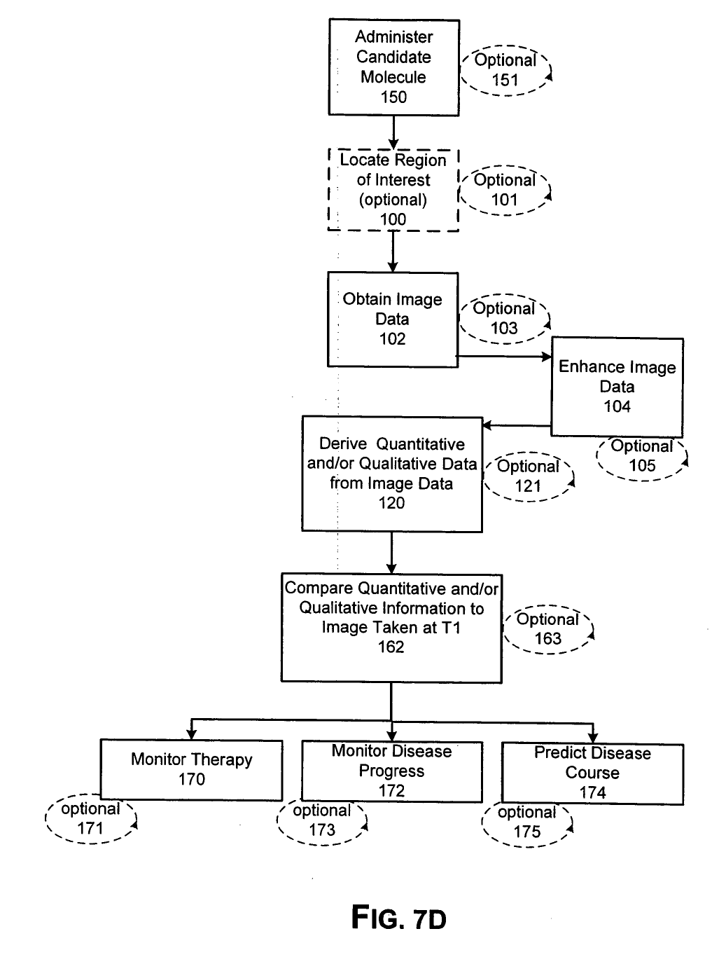

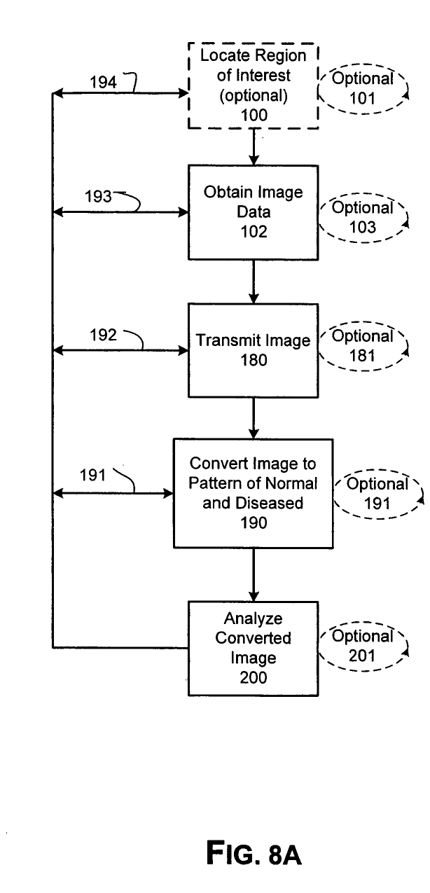

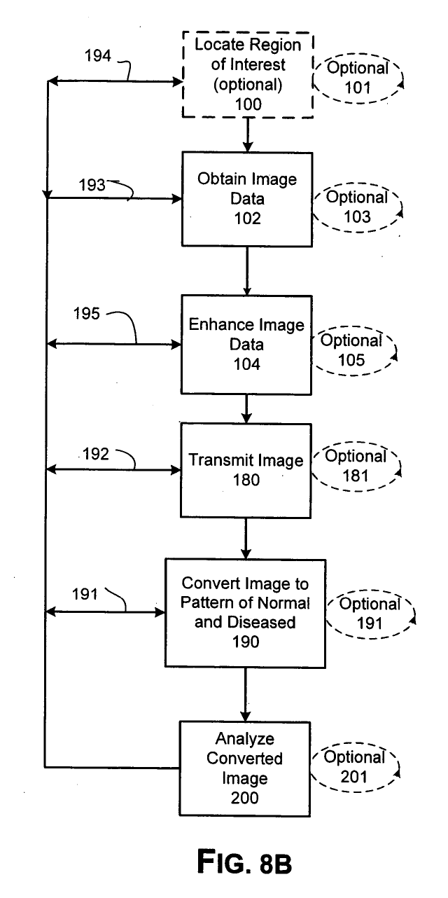

Turning now to FIG. 8A, following the step of extracting image data from the ROI 102, the image can be transmitted 180. Transmission can be to another computer in the network or via the World Wide Web to another network. Following the step of transmitting the image 180, the image is converted to a pattern of normal and diseased tissue 190 Normal tissue includes the undamaged tissue located in the body part selected for study. Diseased tissue includes damaged tissue located in the body part selected for study. Diseased tissue can also include, or refer to, a lack of normal tissue in the body part selected for study. For example, damaged or missing cartilage would be considered diseased tissue. Once the image is converted, it is analyzed 200. FIG. 8B illustrates the process shown in FIG. 8A with the additional step of enhancing image data 104. As will be appreciated by those of skill in the art, this process can be repeated 105 as desired.

As shown in FIG. 8c, the step of transmitting the image 180 illustrated in FIG. 8A is optional and need not be practiced under the invention. As will be appreciated by those of skill in the art, the image can also be analyzed prior to converting the image to a pattern of normal and diseased. FIG. 8D illustrates the process shown in FIG. 8c with the additional step of enhancing image data 104 which is optionally repeated 105, as desired.

As previously described, some or all the processes in FIGS. 8A-D can be repeated one or more times as desired. For example, locating a region of interest 100, obtaining image data 102, enhancing image data 104, transmitting an image 180, converting the image to a pattern of normal and diseased 190, analyzing the converted image 200, can be repeated one or more times if desired, 101, 103, 105, 181, 191, 201 respectively.

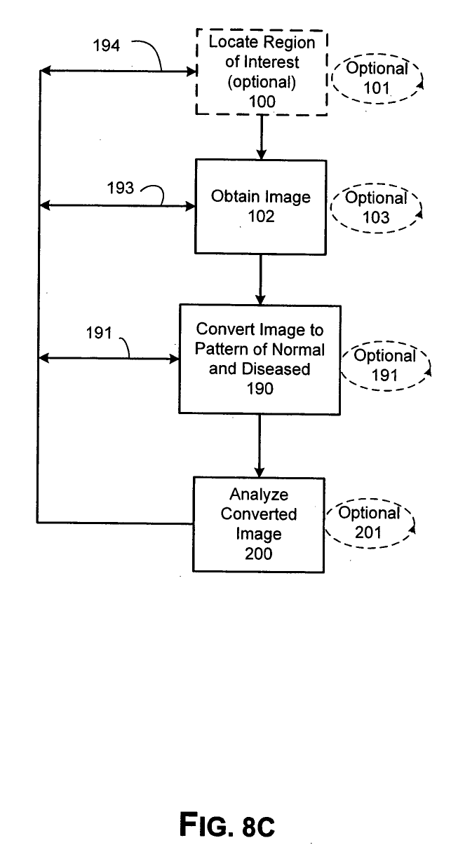

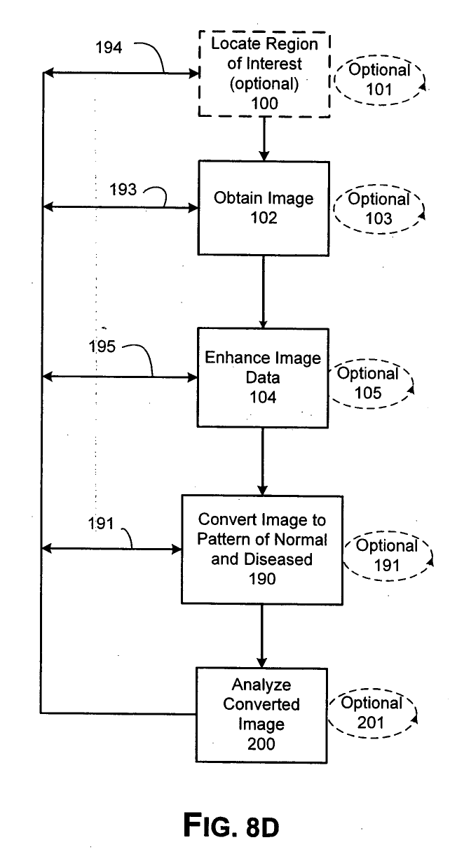

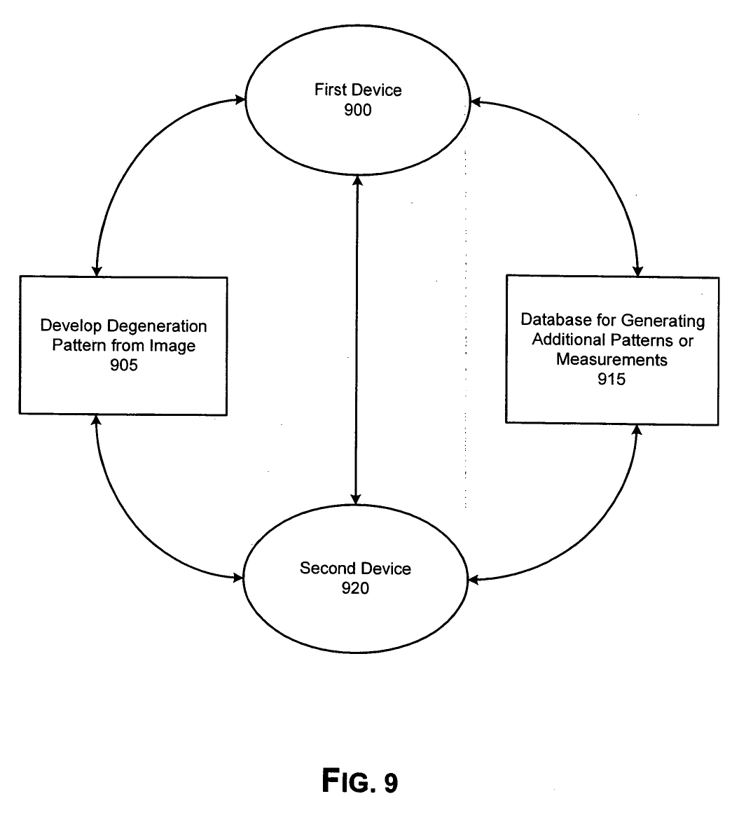

FIG. 9 shows two devices 900, 920 that are connected. Either the first or second device can develop a degeneration pattern from an image of a region of interest 905. Similarly, either device can house a database for generating additional patterns or measurements 915. The first and second devices can communicate with each other in the process of analyzing an image, developing a degeneration pattern from a region of interest in the image, and creating a dataset of patterns or measurements or comparing the degeneration pattern to a database of patterns or measurements. However, all processes can be performed on one or more devices, as desired or necessary.

In this method the electronically generated, or digitized image or portions of the image can be electronically transferred from a transferring device to a receiving device located distant from the transferring device; receiving the transferred image at the distant location; converting the transferred image to a pattern of normal or diseased or abnormal tissue using one or more of the parameters specified in Table 1, Table 2 or Table 3; and optionally transmitting the pattern to a site for analysis. As will be appreciated by those of skill in the art, the transferring device and receiving device can be located within the same room or the same building. The devices can be on a peer-to-peer network, or an intranet. Alternatively, the devices can be separated by large distances and the information can be transferred by any suitable means of data transfer, including the World Wide Web and ftp protocols.

Alternatively, the method can comprise electronically transferring an electronically-generated image or portions of an image of a bone or a joint from a transferring device to a receiving device located distant from the transferring device; receiving the transferred image at the distant location; converting the transferred image to a degeneration pattern or a pattern of normal or diseased or abnormal tissue using one or more of the parameters specified in Table 1, Table 2 or Table 3; and optionally transmitting the degeneration pattern or the pattern of normal or diseased or abnormal tissue to a site for analysis.

Another aspect of the invention is a kit for aiding in assessing the condition of a bone or a joint of a subject, which kit comprises a software program, which when installed and executed on a computer reads a degeneration pattern or a pattern of normal or diseased or abnormal tissue derived using one or more of the parameters specified in Table 1, Table 2 or Table 3 presented in a standard graphics format and produces a computer readout. The kit can further include a database of measurements for use in calibrating or diagnosing the subject. One or more databases can be provided to enable the user to compare the results achieved for a specific subject against, for example, a wide variety of subjects, or a small subset of subjects having characteristics similar to the subject being studied.

A system is provided that includes (a) a device for electronically transferring a degeneration pattern or a pattern of normal, diseased or abnormal tissue for the bone or the joint to a receiving device located distant from the transferring device; (b) a device for receiving said pattern at the remote location; (c) a database accessible at the remote location for generating additional patterns or measurements for the bone or the joint of the human wherein the database includes a collection of subject patterns or data, for example of human bones or joints, which patterns or data are organized and can be accessed by reference to characteristics such as type of joint, gender, age, height, weight, bone size, type of movement, and distance of movement; (d) optionally a device for transmitting the correlated pattern back to the source of the degeneration pattern or pattern of normal, diseased or abnormal tissue.

Thus, the methods and systems described herein make use of collections of data sets of measurement values, for example measurements of bone structure and/or bone mineral density from x-ray images. Records can be formulated in spreadsheet-like format, for example including data attributes such as date of x-ray, patient age, sex, weight, current medications, geographic location, etc. The database formulations can further comprise the calculation of derived or calculated data points from one or more acquired data points, typically using the parameters listed in Tables 1, 2 and 3 or combinations thereof. A variety of derived data points can be useful in providing information about individuals or groups during subsequent database manipulation, and are therefore typically included during database formulation. Derived data points include, but are not limited to the following: (1) maximum value, e.g. bone mineral density, determined for a selected region of bone or joint or in multiple samples from the same or different subjects; (2) minimum value, e.g. bone mineral density, determined for a selected region of bone or joint or in multiple samples from the same or different subjects; (3) mean value, e.g. bone mineral density, determined for a selected region of bone or joint or in multiple samples from the same or different subjects; (4) the number of measurements that are abnormally high or low, determined by comparing a given measurement data point with a selected value; and the like. Other derived data points include, but are not limited to the following: (1) maximum value of a selected bone structure parameter, determined for a selected region of bone or in multiple samples from the same or different subjects; (2) minimum value of a selected bone structure parameter, determined for a selected region of bone or in multiple samples from the same or different subjects; (3) mean value of a selected bone structure parameter, determined for a selected region of bone or in multiple samples from the same or different subjects; (4) the number of bone structure measurements that are abnormally high or low, determined by comparing a given measurement data point with a selected value; and the like. Other derived data points will be apparent to persons of ordinary skill in the art in light of the teachings of the present specification. The amount of available data and data derived from (or arrived at through analysis of) the original data provides an unprecedented amount of information that is very relevant to management of bone-related diseases such as osteoporosis. For example, by examining subjects over time, the efficacy of medications can be assessed.

Measurements and derived data points are collected and calculated, respectively, and can be associated with one or more data attributes to form a database. The amount of available data and data derived from (or arrived at through analysis of) the original data provide provides an unprecedented amount of information that is very relevant to management of musculoskeletal-related diseases such as osteoporosis or arthritis. For example, by examining subjects over time, the efficacy of medications can be assessed.

Data attributes can be automatically input with the electronic image and can include, for example, chronological information (e.g., DATE and TIME). Other such attributes can include, but are not limited to, the type of imager used, scanning information, digitizing information and the like. Alternatively, data attributes can be input by the subject and/or operator, for example subject identifiers, i.e. characteristics associated with a particular subject. These identifiers include but are not limited to the following: (1) a subject code (e.g., a numeric or alpha-numeric sequence); (2) demographic information such as race, gender and age; (3) physical characteristics such as weight, height and body mass index (BMI); (4) selected aspects of the subject's medical history (e.g., disease states or conditions, etc.); and (5) disease-associated characteristics such as the type of bone disorder, if any; the type of medication used by the subject. In the practice of the present invention, each data point would typically be identified with the particular subject, as well as the demographic, etc. characteristic of that subject.

Other data attributes will be apparent to persons of ordinary skill in the art in light of the teachings of the present specification. (See, also, WO 02/30283, incorporated by reference in its entirety herein).

Thus, data (e.g., bone structural information or bone mineral density information or articular information) is obtained from normal control subjects using the methods described herein. These databases are typically referred to as “reference databases” and can be used to aid analysis of any given subject's x-ray image, for example, by comparing the information obtained from the subject to the reference database. Generally, the information obtained from the normal control subjects will be averaged or otherwise statistically manipulated to provide a range of “normal” measurements. Suitable statistical manipulations and/or evaluations will be apparent to those of skill in the art in view of the teachings herein. The comparison of the subject's x-ray information to the reference database can be used to determine if the subject's bone information falls outside the normal range found in the reference database or is statistically significantly different from a normal control.

Data obtained from images, as described above, can be manipulated, for example, using a variety of statistical analyses to produce useful information. Databases can be created or generated from the data collected for an individual, or for a group of individuals, over a defined period of time (e.g., days, months or years), from derived data, and from data attributes.

For example, data can be aggregated, sorted, selected, sifted, clustered and segregated by means of the attributes associated with the data points. A number of data mining software exist which can be used to perform the desired manipulations.

Relationships in various data can be directly queried and/or the data analyzed by statistical methods to evaluate the information obtained from manipulating the database.

For example, a distribution curve can be established for a selected data set, and the mean, median and mode calculated therefor. Further, data spread characteristics, e.g., variability, quartiles, and standard deviations can be calculated.

The nature of the relationship between any variables of interest can be examined by calculating correlation coefficients. Useful methods for doing so include, but are not limited to: Pearson Product Moment Correlation and Spearman Rank Correlation. Analysis of variance permits testing of differences among sample groups to determine whether a selected variable has a discernible effect on the parameter being measured.

Non-parametric tests can be used as a means of testing whether variations between empirical data and experimental expectancies are attributable to chance or to the variable or variables being examined. These include the Chi Square test, the Chi Square Goodness of Fit, the 2×2 Contingency Table, the Sign Test and the Phi Correlation Coefficient. Other tests include z-scores, T-scores or lifetime risk for arthritis, cartilage loss or osteoporotic fracture.

There are numerous tools and analyses available in standard data mining software that can be applied to the analyses of the databases that can be created according to this invention. Such tools and analysis include, but are not limited to, cluster analysis, factor analysis, decision trees, neural networks, rule induction, data driven modeling, and data visualization. Some of the more complex methods of data mining techniques are used to discover relationships that are more empirical and data-driven, as opposed to theory driven, relationships.

Statistical significance can be readily determined by those of skill in the art. The use of reference databases in the analysis of x-ray images facilitates that diagnosis, treatment and monitoring of bone conditions such as osteoporosis.

For a general discussion of statistical methods applied to data analysis, see Applied Statistics for Science and Industry, by A. Romano, 1977, Allyn and Bacon, publisher.

The data is preferably stored and manipulated using one or more computer programs or computer systems. These systems will typically have data storage capability (e.g., disk drives, tape storage, optical disks, etc.). Further, the computer systems can be networked or can be stand-alone systems. If networked, the computer system would be able to transfer data to any device connected to the networked computer system for example a medical doctor or medical care facility using standard e-mail software, a central database using database query and update software (e.g., a data warehouse of data points, derived data, and data attributes obtained from a large number of subjects). Alternatively, a user could access from a doctor's office or medical facility, using any computer system with Internet access, to review historical data that can be useful for determining treatment.

If the networked computer system includes a World Wide Web application, the application includes the executable code required to generate database language statements, for example, SQL statements. Such executables typically include embedded SQL statements. The application further includes a configuration file that contains pointers and addresses to the various software entities that are located on the database server in addition to the different external and internal databases that are accessed in response to a user request. The configuration file also directs requests for database server resources to the appropriate hardware, as can be necessary if the database server is distributed over two or more different computers.

As a person of skill in the art will appreciate, one or more of the parameters specified in Table 1, Table 2 and Table 3 can be used at an initial time point T₁ to assess the severity of a bone disease such as osteoporosis or arthritis. The patient can then serve as their own control at a later time point T₂, when a subsequent measurement using one or more of the same parameters used at T₁ is repeated.

A variety of data comparisons can be made that will facilitate drug discovery, efficacy, dosing, and comparisons. For example, one or more of the parameters specified in Table 1, Table 2 and Table 3 can be used to identify lead compounds during drug discovery. For example, different compounds can be tested in animal studies and the lead compounds with regard to highest therapeutic efficacy and lowest toxicity, e.g. to the bone or the cartilage, can be identified. Similar studies can be performed in human subjects, e.g. FDA phase I, II or III trials. Alternatively, or in addition, one or more of the parameters specified in Table 1, Table 2 and Table 3 can be used to establish optimal dosing of a new compound. It will be appreciated also that one or more of the parameters specified in Table 1, Table 2 and Table 3 can be used to compare a new drug against one or more established drugs or a placebo.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

What is claimed is:

1. A method for analyzing at least one of bone mineral density, bone structure and surrounding tissue comprising:
a. obtaining an image of a subject;
b. locating a region of interest on the image;
c. obtaining data from the region of interest; and
d. deriving data selected from the group of qualitative and quantitative from the image data obtained at step c.

2. The method of claim 1 further including the step of enhancing image data extracted from the region of interest.

3. The method of claim 1 wherein the subject is a mammal.

4. The method of claim 1 wherein the subject is a human.

5. The method of claim 1 wherein the subject is a horse.

6. The method of claim 1 wherein the step of obtaining image data includes obtaining data from a measured parameter selected from the group consisting of: bone parameters, cartilage parameters, cartilage defect parameters, cartilage disease parameters, area parameters, and volume parameters.

7. The method of claim 1 wherein the step of obtaining image data includes extracting data from a measured parameter selected from the group consisting of:
a presence or absence of bone marrow edema;
a volume of bone marrow edema;
a volume of bone marrow edema normalized by at least one of width, area, size, and volume;
a presence or absence of osteophytes;
a presence or absence of subchondral cysts;
a presence or absence of subchondral sclerosis;
a volume of osteophytes; a volume of subchondral cysts;
a volume of subchondral sclerosis;
an area of bone marrow edema; an area of osteophytes;
an area of subchondral cysts;
an area of subchondral sclerosis;
a depth of bone marrow edema;
a depth of osteophytes;
a depth of subchondral cysts;
a depth of subchondral sclerosis;
at least one of a volume, area, and depth of at least one of an osteophytes, subchondral cysts, subchondral sclerosis wherein the at least one of volume, area, and depth is normalized by at least one of width, area, size, volume a bone proximal to at least one of the osteophyte, subchondral cyst, or subchondral sclerosis;
a presence or absence of meniscal tear;
a presence or absence of cruciate ligament tear;
a presence or absence of collateral ligament tear;
a volume of menisci;
a ratio of volume of normal to at least one of torn, damaged and degenerated meniscal tissue;
a ratio of surface area of normal to at least one of torn, damaged and degenerated meniscal tissue;
a ratio of surface area of normal to at least one of torn, damaged and degenerated meniscal tissue to total joint or cartilage surface area;
a ratio of surface area of at least one of torn, damaged and degenerated meniscal tissue to a total surface area of at least one of joint and cartilage;
a size ratio of opposing articular surfaces;
a meniscal subluxation/dislocation in millimeters;
an index combining different articular parameters;
a 3D surface contour information of subchondral bone;
an actual or predicted knee flexion angle during gait cycle;
a predicted knee rotation during gait cycle;
a predicted knee displacement during gait cycle;
a predicted load bearing line on cartilage surface during gait cycle and measurement of distance between load bearing line and at least one of cartilage defect and diseased cartilage;
a predicted load bearing area on cartilage surface during gait cycle and measurement of distance between load bearing area and at least one of cartilage defect and diseased cartilage;
a predicted load bearing line on cartilage surface during standing or different degrees of knee flexion and extension and measurement of distance between load bearing line and at least one of cartilage defect and diseased cartilage;
a predicted load bearing area on cartilage surface during standing or different degrees of knee flexion and extension and measurement of distance between load bearing area and at least one of cartilage defect and diseased cartilage;
a ratio of load bearing area to area of at least one of cartilage defect and diseased cartilage;
a percentage of load bearing area affected by cartilage disease;
a location of cartilage defect within load bearing area;
a load applied to cartilage defect, area of diseased cartilage; and
a load applied to cartilage adjacent to at least one of cartilage defect and area of diseased cartilage.

8. The method of claim 7 wherein the index combining different articular parameters includes:
a presence or absence of cruciate or collateral ligament tear in the subject,
a body mass index for the subject,
a weight for the subject, or
a height for the subject.

9. The method of claim 1 wherein the image data is obtained of a hip and the step of obtaining image data includes extracting data from a measured parameter selected from the group consisting of:
microarchitecture parameters on structures parallel to stress lines;
microarchitecture parameters on structures perpendicular to stress lines;
geometry;
shaft angle;
neck angle;
diameter of femur neck;
hip axis length;
largest cross-section of femur head;
average thickness of cortical within at least one ROI;
standard deviation of cortical thickness within at least one ROI;
maximum thickness of cortical within at least one ROI;
minimum thickness of cortical within at least one ROI; and
hip joint space width.

10. The method of claim 1 wherein the image data is obtained from a region of a spine and the step of obtaining image data includes extracting data from a measured parameter selected from the group consisting of:
microarchitecture parameters on vertical structures;
microarchitecture parameters on horizontal structures;
geometry;
superior endplate cortical thickness;
inferior endplate cortical thickness;
anterior vertebral wall cortical thickness;
posterior vertebral wall cortical thickness;
superior aspect of pedicle cortical thickness;
inferior aspect of pedicle cortical thickness;
vertebral height;
vertebral diameter;
pedicle thickness;
maximum vertebral height;
minimum vertebral height;
average vertebral height;
anterior vertebral height;
medial vertebral height;
posterior vertebral height;
maximum inter-vertebral height;
minimum inter-vertebral height; and
average inter-vertebral height.

11. The method of claim 1 wherein the image data is obtained from a region of a knee and the step of obtaining image data includes extracting data from a measured parameter selected from the group consisting of:
average medial joint space width;
minimum medial joint space width;
maximum medial joint space width;
average lateral joint space width;
minimum lateral joint space width; and
maximum lateral joint space width.

12. The method of claim 1 wherein the step of obtaining image data includes extracting bone parameters selected from the group consisting of:
stainless steel equivalent thickness wherein the stainless steel equivalent thickness is determined as the average gray value of the region of interest expressed as thickness of stainless steel with equivalent intensity;
trabecular contrast wherein the trabecular contrast is determined as one of the trabecular equivalent thickness and marrow equivalent thickness;
fractal dimension;
Fourier spectral analysis wherein the Fourier spectral analysis is determined as one of a mean transform coefficient absolute value and a mean spatial first moment;
predominant orientation of spatial energy spectrum;
at least one of trabecular area and total area;
trabecular perimeter;
trabecular distance transform;
marrow distance transform;
trabecular distance transform regional maxima values;
marrow distance transform regional maxima values;
star volume;
trabecular bone pattern factor;
connected skeleton count or trees (T);
node count (N);
segment count (S);
node-to-node segment count (NN);
node-to-free-end segment count (NF);
node-to-node segment length (NNL)
node-to-free-end segment length (NFL);
free-end-to-free-end segment length (FFL);
node-to-node total struts length (NN.TSL);
free-end-to-free-ends total struts length (FF.TSL);
total struts length (TSL);
FF.TSL/TSL;
NN.TSL/TSL;
loop count (Lo);
loop area;
mean distance transform values for each connected skeleton;
mean distance transform values for each segment (Tb.Th);
mean distance transform values for each node-to-node segment (Tb.Th.NN);
mean distance transform values for each node-to-free-end segment (Tb.Th.NF);
orientation of each segment;
angle of each segment;
angle between segments;
length-thickness ratios (NNL/Tb.Th.NN) and (NFL/Tb.Th.NF); and
interconnectivity index (ICI) ICI=(N*NN)/(T*(NF+1));

13. The method of claim 12 wherein total bone parameter factor is (P1−P2)/(A1−A2)
further wherein P1 and A1 are the perimeter length and trabecular bone area before dilation and P2 and A2 corresponding values after a single pixel dilation, measure of connectivity

14. The method of claim 1 wherein the step of obtaining image data from the region of interest includes extracting at least one of cartilage parameters, cartilage defect parameters, and diseased cartilage parameters, wherein the data extracted is selected from the group consisting of:
total cartilage volume;
focal cartilage volume;
a cartilage thickness distribution or thickness map;
mean cartilage thickness over substantially total surface;
mean cartilage thickness in focal area;
median cartilage thickness;
maximum cartilage thickness;
minimum cartilage thickness;
3D cartilage surface information;
cartilage curvature analysis;
volume of cartilage defect/diseased cartilage;
depth of cartilage defect/diseased cartilage;
area of cartilage defect/diseased cartilage;
at least one of 2D and 3D location of cartilage defect/diseased cartilage in the articular surface;
at least one of 2D and 3D location of cartilage defect/diseased cartilage in relationship to weight-bearing area;
a ratio of at least two of diameter of cartilage defect, diameter of diseased cartilage, and thickness of surrounding normal cartilage;
a ratio of at least two of depth of cartilage defect, depth of diseased cartilage and thickness of surrounding normal cartilage;
a ratio of at least two of volume of cartilage defect, volume of diseased cartilage and thickness of surrounding normal cartilage;
a ratio of at least two of surface area of cartilage defect, surface area of diseased cartilage and total joint surface area; and
a ratio of at least two of volume of cartilage defect, volume of diseased cartilage and total cartilage volume.

15. The method of claim 1 wherein at least one step is performed automatically.

16. The method of claim 1 wherein at least one step is performed semi-automatically.

17. The method of claim 1 wherein at least one of the steps is performed on a first computer.

18. The method of claim 1 wherein at least one of the steps is performed on a first computer and at least one of the steps is performed on a second computer.

19. The method of claim 18 wherein the first computer and the second computer are connected by one of a peer to peer network, direct link, intranet, and internet.

20. The method of claim 1 wherein the step of locating a region of interest is repeated.

21. The method of claim 1 wherein the step of obtaining image data from a region of interest is repeated.

22. The method of claim 1 wherein at least one of the image and data is converted to a 2D pattern.

23. The method of claim 22 wherein the 2D pattern is evaluated.

24. The method of claim 22 wherein the 2D pattern is converted to a 3D pattern.

25. The method of claim 23 wherein the 2D pattern is converted to a 3D pattern.

26. The method of claim 22 wherein the 2D pattern is converted to a 4D pattern.

27. The method of claim 23 wherein the 2D pattern is converted to a 4D pattern.

28. The method of claim 25 wherein the 3D pattern is converted to a 4D pattern.

29. The method of claim 1 wherein at least one of the image and data is converted to a 3D pattern.

30. The method of claim 29 wherein the 3D pattern is evaluated.

31. The method of claim 29 wherein the 3D pattern is converted to a 2D pattern.

32. The method of claim 31 wherein the 3D pattern is converted to a 2D pattern.

33. The method of claim 29 wherein the 3D pattern is converted to a 4D pattern.

34. The method of claim 30 wherein the 3D pattern is converted to a 4D pattern.

35. The method of claim 31 wherein the 2D pattern is converted to a 4D pattern.

36. The method of claim 1 wherein the at least one of the image and data is converted to a 4D pattern.

37. The method of claim 36 wherein the 4D pattern is evaluated.

38. The method of claim 1 further comprising the step of administering a candidate agent.

39. The method of claim 38 wherein the candidate agent is at least one agent selected from the group consisting of: substance administered to a subject, substance ingested by a subject, molecules, pharmaceuticals, biopharmaceuticals, agropharmaceuticals.

40. The method of claim 1 further comprising the step of comparing the data obtained to a database.

41. The method of claim 1 further comprising the step of comparing the data obtained to a subset of a database.

42. The method of claim 1 further comprising the step of comparing at least one of quantitative data and qualitative data to an image taken at T₁.

43. The method of claim 1 further comprising the step of comparing at least one of the quantitative data and qualitative data to an image taken prior to the image under analysis.

44. The method of claim 1 further comprising the step of comparing the at least one of the quantitative data and qualitative data to an image taken at Tn.

45. The method of claim 1 further comprising the step of transmitting at least one of the image and data.

46. The method of claim 45 further comprising the step of analyzing the converted image.

47. The method of claim 46 wherein the step of analyzing the converted image occurs at least at one of prior to transmitting the image or after transmitting the image.

48. The method of claim 1 wherein the image is converted to at least one of a pattern of normal, diseased, and normal and diseased.

49. The method of claim 1 wherein obtaining image data includes measuring at least one of microarchitecture and macroanatomical structures.

50. The method of claim 49 further comprising measuring the average density.

51. The method of claim 50 wherein the average density measurement includes a calibrated density of the region of interest.

52. The method of claim 49 further comprising measuring microanatomical structures on at least one of dental, spine, hip, knee and bone core x-rays.

53. The method of claim 52 further comprising measuring at least one of:
calibrated density of extracted structures;
calibrated density of background;
average intensity of extracted structures;
average intensity of background area wherein the background area includes non-extracted structures;
structural contract wherein structural contrast is an average intensity of extracted structures divided by an average intensity of a background;
calibrated structural contrast wherein calibrated structural contrast is a calibrated density of extracted structures divided by a calibrated density of a background;
total area of extracted structures;
total area of a region of interest;
an area of extracted structures normalized by a total area of a region of interest;
a boundary length of an extracted area normalized by a total are of a region of interest;
a number of structures normalized by an area of a region of interest;
a trabecular bone pattern factor;
a measurement of concavity and convexity of structures;
a star volume of extracted structures;
a star volume of background; and
a number of loops normalized by an area of a region of interest.

54. The method of claim 49 further comprising measuring a distance transform of extracted structures.

55. The method of claim 54 wherein the measurement on the distance transform of extracted structures further comprises one or more of:
an average regional maximum thickness;
a standard deviation of regional maximum thickness;
a largest value of regional maximum thickness; and
a median regional maximum thickness.

56. A kit for aiding in the assessment of the condition of at least one of a bone and joint comprising:
a software program which reads at least one of a degeneration pattern, a pattern of normal tissue, a pattern of abnormal tissue, and a pattern of diseased tissue.

57. The kit of claim 56 further comprising a database of measurements for comparison to the at least one of degeneration pattern, pattern of normal tissue, pattern of abnormal tissue, and pattern of diseased tissue.

58. The kit of claim 56 further comprising a subset of a database of measurements for comparison to the at least one of degeneration pattern, pattern of normal tissue, pattern of abnormal tissue, and pattern of diseased tissue.

59. An automated method of using an imaging marker comprising:
obtaining image data;
obtaining data from the image data wherein the data obtained is at least one of quantitative and qualitative data; and
administering an agent.

60. The method of claim 59 wherein the automated method is used for at least one of drug discovery, diagnosis, disease staging, disease monitoring, disease management, prognostication, therapy monitoring, drug efficacy monitoring, and disease prediction.

61. A semi-automated method of using an imaging marker comprising:
obtaining image data;
obtaining data from the image data wherein the data obtained is at least one of quantitative and qualitative data; and
administering an agent.

62. The method of claim 61 wherein the automated method is used for at least one of drug discovery, diagnosis, disease staging, disease monitoring, disease management, prognostication, therapy monitoring, drug efficacy monitoring, and disease prediction.

63. A system for monitoring the efficacy of an agent comprising:
administering an agent to a subject;
obtaining image data; and
obtaining data from the image data wherein the data obtained is at least one of quantitative and qualitative data.

64. A system for drug discovery comprising:
administering an agent to a subject;
obtaining image data; and
obtaining data from the image data wherein the data obtained is at least one of quantitative and qualitative data.

65. A system for diagnosing a disease comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

66. A system for diagnosing a disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

67. A system for diagnosing a disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

68. A system for determining disease staging comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

69. A system for determining disease staging comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

70. A system for determining disease staging comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

71. A system for monitoring disease progression comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

72. A system for monitoring disease progression comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

73. A system for monitoring disease progression comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

74. A system for managing a disease comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

75. A system for managing a disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

76. A system for managing a disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

77. A system for disease prognostication comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

78. A system for disease prognostication comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

79. A system for disease prognostication comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

80. A system for predicting disease comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

81. A system for predicting disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

82. A system for predicting disease comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

83. A system for monitoring therapy comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

84. A system for monitoring therapy comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

85. A system for monitoring therapy comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.

86. A system for randomizing a group of patients comprising:
obtaining image data;
obtaining data from the image data wherein the data obtained is at least one of quantitative and qualitative data; and
comparing the at least one of quantitative and qualitative data to a database of at least quantitative and qualitative data obtained from a group of subjects.

87. A system for randomizing a group of patients comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time T1.

88. A system for randomizing a group of patients comprising:
obtaining image data;
obtaining data from the image data wherein the data obtained is at least one of quantitative and qualitative data; and
comparing the at least one of quantitative and qualitative data to a at least one of quantitative and qualitative data obtained from the subject at time Tn.
